(19) United States
(12) Patent Application Publication
(10) Pub. No.: US 2025/0090049 A1
(43) Pub. Date: Mar. 20, 2025

(54) WEARABLE BIOSENSOR DEVICE AND METHOD FOR DETECTION AND MEASURMENT OF BIO-MOLECULES AND BIO-PARTICLES

(71) Applicant: SenseSi Technology Inc., Calgary (CA)

(72) Inventors: Amir SANATI-NEZHAD, Calgary (CA); Jaideep Singh BAINS, Calgary (CA); Shaghayegh SHAJARI, Calgary (CA); Razieh SALAHANDISH, Calgary (CA); David George ROSENEGGER, Calgary (CA)

(21) Appl. No.: 18/294,405

(22) PCT Filed: Aug. 4, 2022
(86) PCT No.: PCT/CA2022/051189

§ 371 Date: Feb. 1, 2024

Related U.S. Application Data
(60) Provisional application No. 63/229,216, filed on Aug. 4, 2021.

(51) Int. Cl. A61B 5/145; A61B 5/00; A61B 5/1477; A61B 5/16; A61B 10/00

(52) U.S. Cl. CPC A61B 5/14517; A61B 5/14546; A61B 5/1477; A61B 5/165; A61B 5/681; A61B 10/0064; A61B 2010/009; A61B 2560/0462

(57) ABSTRACT

A wearable apparatus for collecting sweat from a subject for stress monitoring. The apparatus comprises a fluid collection device configured for direct contact with the subject's skin when the wearable apparatus is in use. The fluid collection device comprises a main channel configured to collect sweat from the subject's skin, and a plurality of inlet channels in communication with the main channel, and at least one vent. A plurality of inlet ports are each uniquely associated with one of the inlet channels for storing a sample of the collected sweat and preserving molecular contents of the stored sweat. The apparatus may comprise a plurality of collection chambers to absorb the sweat for later analysis, and/or a biosensor configured to sense one or more target biomolecules and/or bioparticles in the collected sweat. A wearable sweat collection and analysis system may include a wearable apparatus as described, and a potentiostat readout device.

### CROSS-REFERENCE TO RELATED APPLICATIONS

The present application claims priority to U.S. Patent Application Ser. No. 63/229,216 filed Aug. 4, 2021, which is hereby incorporated by reference in its entirety.

### FIELD

The present disclosure relates generally to wearable devices, and to related methods of measuring health parameters, for example by fluid collection.

### BACKGROUND

When a person feels threatened, or stressed, their nervous system responds by releasing a flood of hormones, including adrenaline and cortisol, which rouse the body for emergency action. Numerous effects can be observed on the brain, cardiovascular system, joints and muscles, immune system, skin, gut and reproductive system. For example, the heart pounds faster, muscles tighten, blood pressure rises, breath quickens, and senses become sharper.

Monitoring a person's stress level on a routine, and sometimes continuous, basis is becoming increasingly important. One way to do this is by measuring a defined characteristic as an indication of normal processes or responses to interventions. Biomarkers and bioparticles are molecules that are present in biological fluids, the concentrations of which vary with the stress level of the subject. Current approaches for biomarker measurement, for example in relation to stress, can be expensive, difficult and/or require complicated equipment.

Improvements in devices and approaches to measuring health parameters such as stress are desirable.

### SUMMARY

In an aspect, the present disclosure provides a wearable apparatus for collecting an excretion, such as sweat, from a subject for stress monitoring. The excretion may include one or more of sweat, oils, or volatile compounds released from the glands into the air. The apparatus comprises a fluid collection device configured for direct contact with the subject's skin when the wearable apparatus is in use. The fluid collection device comprises a main channel configured to collect sweat from the surface or a sublayer or a subsurface of the subject's skin, and a plurality of inlet channels in communication with the main channel. The fluid collection device further comprises a plurality of inlet ports. Each of the plurality of inlet ports is uniquely associated with one of the plurality of inlet channels, for storing a sample of the collected sweat and preserving molecular contents of the stored sweat. The fluid collection device further comprises at least one vent configured to draw air bubbles out of the sweat collected in the main channel, to assist in sweat collection and preserving the molecular contents of the stored sweat.

In another aspect, the present disclosure provides a wearable apparatus further comprising a plurality of collection chambers configured to collect and absorb the collected sweat for storage for later analysis. The plurality of collection chambers may comprise removable fibers for collecting and absorbing the collected sweat for storage for removal for later analysis.

In another aspect, the present disclosure provides a wearable apparatus further comprising a biosensor in communication with the fluid collection device and configured to sense one or more biomarkers, such as target biomolecules and/or bioparticles in the collected sweat. The biosensor may be printed on the wearable apparatus so as to sense the collected sweat from the main channel.

In another aspect, the present disclosure provides a wearable sweat collection and analysis system comprising a wearable apparatus as described, and further comprising a potentiostat readout device. The potentiostat readout device is in communication with the biosensor and configured to record electrical signals from the biosensor and to analyze the signals to enable digital monitoring of the sweat, and optionally transmit the results.

In another aspect, the present disclosure provides a wearable apparatus for collecting sweat from a subject to enable monitoring of at least one health parameter using a biomarker.

In an implementation, the wearable apparatus comprises a smart patch configured for sensing of biomarkers as feedback for controlled delivery of a drug, for example through a dermal patch or remotely via another delivery method. In another implementation, the wearable apparatus comprises a smart wound dressing to detect infections etc.

### BRIEF DESCRIPTION OF THE DRAWINGS

Embodiments of the present disclosure will now be described, by way of example only, with reference to the attached Figures.

FIG. 1 is a block diagram of a wearable device or system according to an embodiment of the present disclosure.

FIG. 2 illustrates a wearable device according to an embodiment of the present disclosure and illustrating a main channel, vents, inlet channels and inlet ports.

FIG. 3 illustrates a wearable device for sensing biomarkers in collected sweat according to an embodiment of the present disclosure.

FIG. 4 illustrates three embodiments with different channel inlet and/or inlet port sizes according to an embodiment of the present disclosure.

FIG. 5 illustrates a wearable device for sweat collection according to an embodiment of the present disclosure and illustrating a main channel, a vent, inlet channels, inlet ports and collection chambers.

FIG. 6 illustrates side and top views of a wearable device according to an embodiment of the present disclosure to illustrate example thickness and size.

FIG. 7 illustrates a wearable device according to an embodiment of the present disclosure constructed of a flexible material.

FIGS. 8-22 illustrate different example embodiments of wearable devices of the present disclosure, including different layouts, configurations, thicknesses, numbers of channels, and embodiments in use in various locations such as on a hand, in an armpit and on a forehead.

FIGS. 23-25 illustrate sequential filling of inlet ports and/or collection chambers in wearable devices according to embodiments of the present disclosure.

FIGS. 26-28 illustrate optical microscopy of different portions or segments of a wearable device according to embodiments of the present disclosure.

FIG. 29 illustrates an example implementation of a wearable device according to an embodiment of the present disclosure integrated into a smart watch.

FIGS. 30 and 31 illustrate layers of manufacture of a wearable device according to example embodiments of the present disclosure.

FIGS. 32-48 illustrate experimental results of implementation of wearable devices according to example embodiments of the present disclosure.

### DETAILED DESCRIPTION

The present disclosure provides a device comprising a suitable material and microfluidic wearable design that may enable a reliable and evaporation-free collection and/or storage of human sweat, while optionally concurrently enabling evaporation from vents for continuous flow and monitoring. Embodiments of the present disclosure have a positive impact on the sensitivity, selectivity and compatibility with wearable stress biosensors.

A wearable apparatus is provided for collecting sweat from a subject for stress monitoring. In an implementation, the subject is a person. In another implementation, the subject is an animal. The apparatus comprises a fluid collection device configured for direct contact with the subject's skin when the wearable apparatus is in use. The fluid collection device comprises a main channel configured to collect sweat from the subject's skin, and a plurality of inlet channels in communication with the main channel, and at least one vent. A plurality of inlet ports are each uniquely associated with one of the inlet channels for storing a sample of the collected sweat and preserving molecular contents of the stored sweat. The apparatus may comprise a plurality of collection chambers to absorb the sweat for later analysis, and/or a biosensor configured to sense one or more target biomolecules and/or bioparticles in the collected sweat. A wearable sweat collection and analysis system may include a wearable apparatus as described, and a potentiostat readout device.

Certain terms used in this application and their meaning as used in this context are set forth herein. To the extent a term used herein is not defined herein, it should be given the broadest definition persons in the pertinent art have given that term as reflected in at least one printed publication or issued patent. Further, the present processes are not limited by the usage of the terms shown below, as all equivalents, synonyms, new developments and terms or processes that serve the same or a similar purpose are considered to be within the scope of the present disclosure. Throughout this disclosure, where a range is used, any number between or inclusive of the range is implied.

Different biofluids may be used for stress detection. The use of blood for stress detection is invasive, has longer testing times, and pain and anxiety during sample collection can lead to inaccurate stress response. The use of saliva is non-invasive, but requires sensitive detection, can be affected by the last meal eaten, and includes unstable cortisol at room temperature. The use of urine is non-invasive, but is an unreliable collection method, and is unstable at room temperature. On the other hand, the use of sweat is non-invasive, includes extensive data about health, and the method of collection is relatively simple.

Different biomarkers may be used for stress detection. Dopamine, epinephrine and serotonin are biomarkers that are present in blood and urine, but not in sweat. Cortisol is a biomarker that is present in fairly large concentrations in all four bodily fluids, including sweat. Biosensing is an approach that samples sweat, lactate or other biomarkers from a subject's skin, for example using cellulose fibers or another absorbent material, which may cooperate with a screen printed electrode layer to communicate data with a wearable device. The wearable device could be, for example, a smart watch including a potentiostat, a microcontroller and a battery.

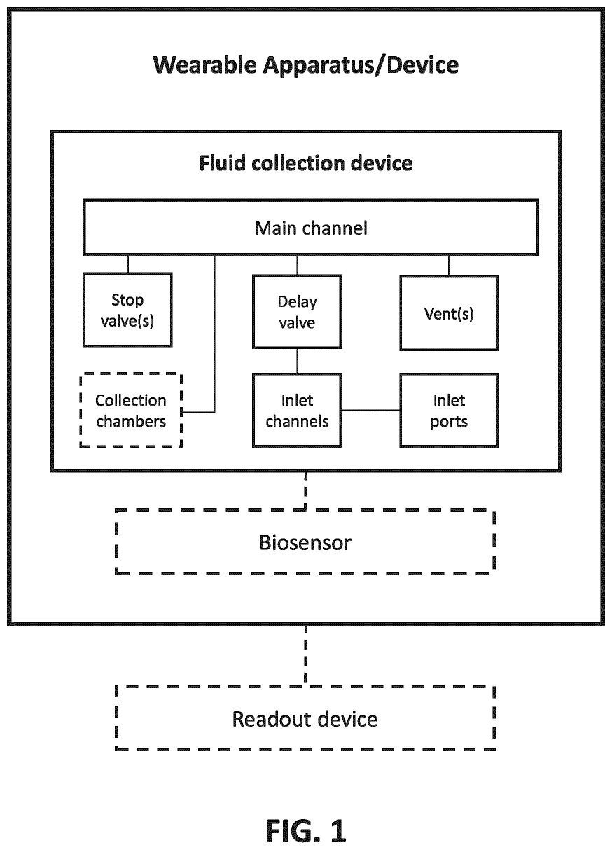

FIG. 1 illustrates a wearable apparatus for stress detection according to an embodiment of the present disclosure. The apparatus of FIG. 1 is a wearable apparatus for collecting sweat from a subject for stress monitoring. The apparatus comprises a fluid collection device configured for direct contact with the subject's skin when the wearable apparatus is in use. The fluid collection device comprises a main channel configured to gather collected sweat from the subject's skin, and a plurality of inlet channels in communication with the main channel. The fluid collection device further comprises a plurality of inlet ports. Each of the plurality of inlet ports is uniquely associated with one of the plurality of inlet channels, for collecting a sample of sweat and preserving molecular contents of the stored sweat.

In an implementation, sweat comes in through the inlet ports from the skin, then through capillary action the sweat is absorbed and channeled via the inlet channels to the main channel. The fluid collection device further comprises at least one stop valve configured to control flow through the device, and/or to measure the timing/rate of flow through the device. The fluid collection device further comprises at least one vent configured to draw air bubbles out of the sweat collected in the main channel, to assist in sweat collection and preserving the molecular contents of stored sweat. Components shown in FIG. 1, for example the at least one vent and/or the at least one stop valve, cooperate to remove air bubbles and to provide a smooth laminar flow down the main channel, which can be beneficial with respect to sensing.

As shown in FIG. 1, a delay valve is provided between the main channel and the inlet channels to control the flow of the liquids. In an embodiment, the delay valve is provided at a junction between the main channel and the inlet channels. In an implementation where the inlet channels are filled separately, the delay valve is configured to delay the flow of liquid from the inlet channel to the main channel until there is appropriate volume to flow through.

The wearable apparatus of FIG. 1 may further comprise a plurality of collection chambers configured to collect and absorb the collected sweat for storage for later analysis. The plurality of collection chambers may comprise removable fibers for collecting and absorbing the collected sweat for storage for removal for later analysis.

The wearable apparatus of FIG. 1 may further comprise a biosensor in communication with the fluid collection device and configured to sense one or more biomarkers, such as target biomolecules and/or bioparticles in the collected sweat. The biosensor may be printed on the wearable apparatus so as to sense the collected sweat from the main channel. In an embodiment including a biosensor, the biosensor may be placed over the main channel, and the main channel may comprise a reaction chamber, such that a chemical reaction or sensing reaction occurs over the main channel. In an example embodiment, the reaction chamber is a specific part or section of the main channel on which the biosensor is provided.

In an example embodiment, the present disclosure provides an approach for collecting sweat with one or more micro fluid patches, and then using sensors built into the patches to detect metabolites in the sweat. In one embodiment, the biomarkers comprise metabolites of stress. The apparatus may provide detection of early states of stress, to enable some kind of intervention to reduce the stress levels. The apparatus may be used to test to determine whether a medication is actually working at alleviating stress, for example by measuring the biomarkers before use of the medication, and comparing it to a measurement after the use of the medication. The apparatus may be used to test any number of other stress reduction interventions such as breathing, meditation, biofeedback, and the like. In another embodiment, the biomarkers can be measured before and after a specified activity, such as a behavioral therapy or relaxation exercise or advanced neurostimulation protocol.

A wearable sweat collection and analysis system according to an embodiment of the present disclosure comprises the wearable apparatus of FIG. 1 as described herein, and a potentiostat readout device. The potentiostat readout device is in communication with the biosensor and configured to record electrical signals from the biosensor and to analyze the signals to enable digital monitoring of the sweat, and optionally transmit the results.

While FIG. 1 is described in relation to sweat collection for stress monitoring, in another embodiment the wearable apparatus is for collecting sweat from a subject to enable monitor at least one health parameter using a biomarker.

A characteristic of a wearable device according to an embodiment of the present disclosure is that the device is relatively easily manufactured, as compared to known designs which are rather complicated and technically intricate, and are expensive and time consuming to produce. Embodiments of the present disclosure provide a wearable device that can be built and manufactured at reasonable costs.

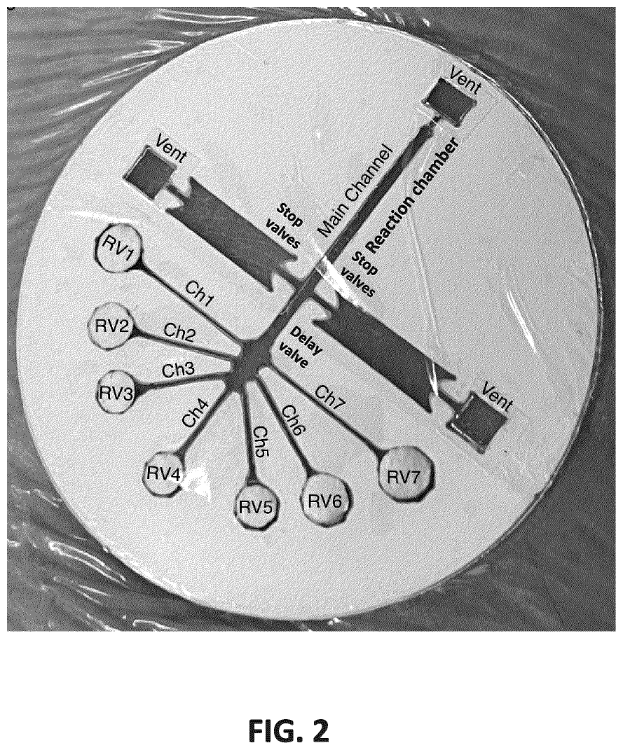

FIG. 2 illustrates a wearable device according to an embodiment of the present disclosure and illustrating a main channel, vents, stop valves, delay valve, inlet channels (labelled Ch1, Ch2, etc.) and inlet ports (labelled RV1, RV2, etc.). The example embodiment of FIG. 2 includes 7 inlet ports, each joined to the main channel by an associated inlet channel and delay valve. Two stop valves, shown in FIG. 2 as rectangular chambers, are connected to the main channel and configured to remove or eliminate air bubbles from the main channel in which sensing occurs. Vents are provided at the end of each rectangular chamber farthest from the main channel, as well as at the end of the main channel opposite the connection points to the inlet ports.

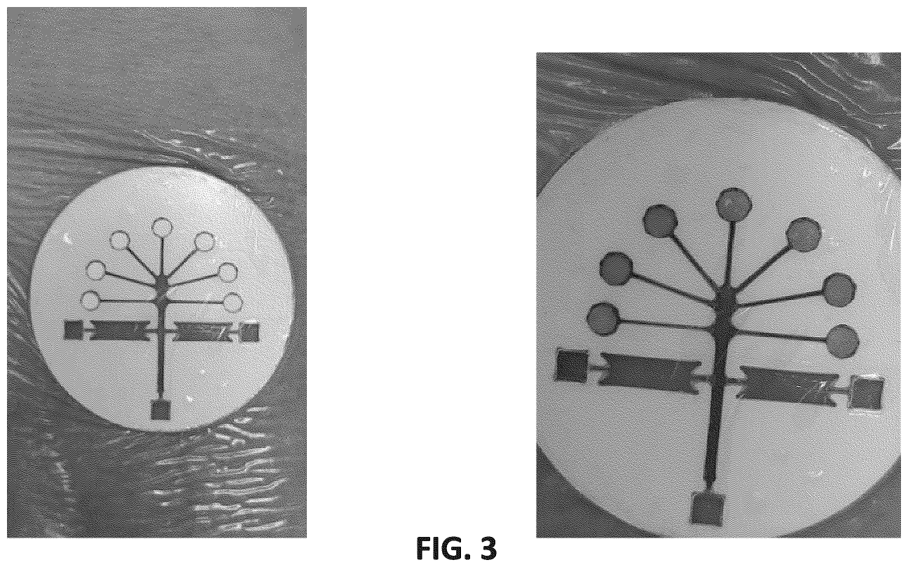

FIG. 3 illustrates a wearable device for sensing biomarkers in collected sweat according to an embodiment of the present disclosure. Similar to FIG. 2, the example embodiment of FIG. 3 includes 7 inlet ports, each joined to the main channel by an associated inlet channel. Two rectangular chambers are connected to the main channel, for the purposes of removing air bubbles before main channel is filled. The rectangular chambers act as stop valves after main channel filling with sweat with hydrophobic surfaces and change in geometry (cross section and angles) in order to keep the liquid in the main channel for sensing and recording data. The stop valves are configured to let the sweat in after specific time for flowing towards outlets and thus providing continuous sweat flow into the device. In an example embodiment, a sensor is provided over the main channel, for example using a printable ink, to enable the wearable device to sense one or more biomarkers associated with stress, for example cortisol. Cortisol circulates in the blood, and peaks typically 60 to 90 minutes after the actual stressor, or stress response. In another example embodiment, the wearable device is configured to sense one or more early appearing metabolites in sweat, to provide data between the stressor and the appearance of cortisol; this can assist in trying to blunt a longer term stress effect, and help avoid as much long term stress load on the body.

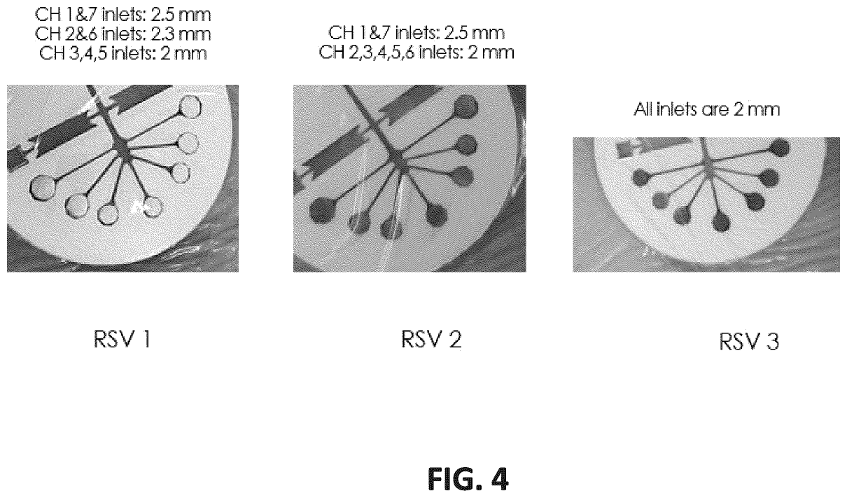

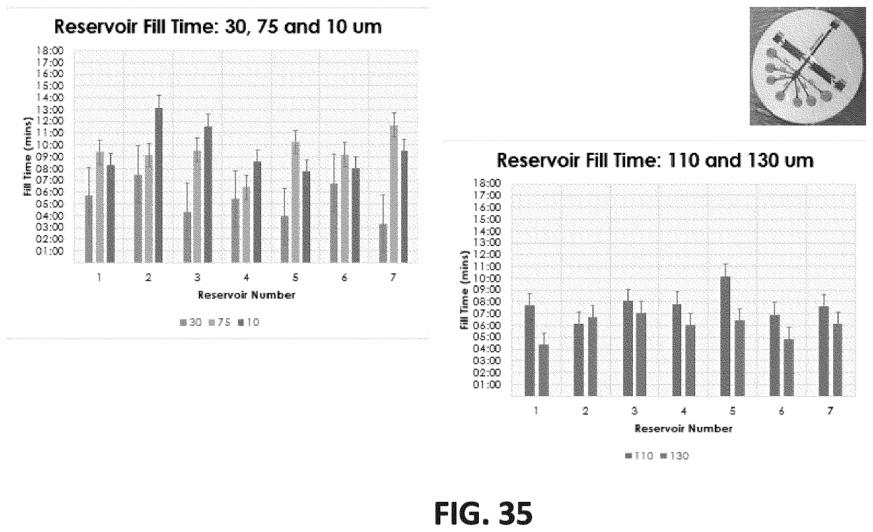

FIG. 4 illustrates three embodiments with different channel inlet and/or inlet port sizes according to an embodiment of the present disclosure. The three different embodiments shown in FIG. 4 illustrate how the inlet channel size may be varied to achieve different results. Results relating to the RSV1, RSV2 and RSV3 embodiments are illustrated later in relation to FIG. 35.

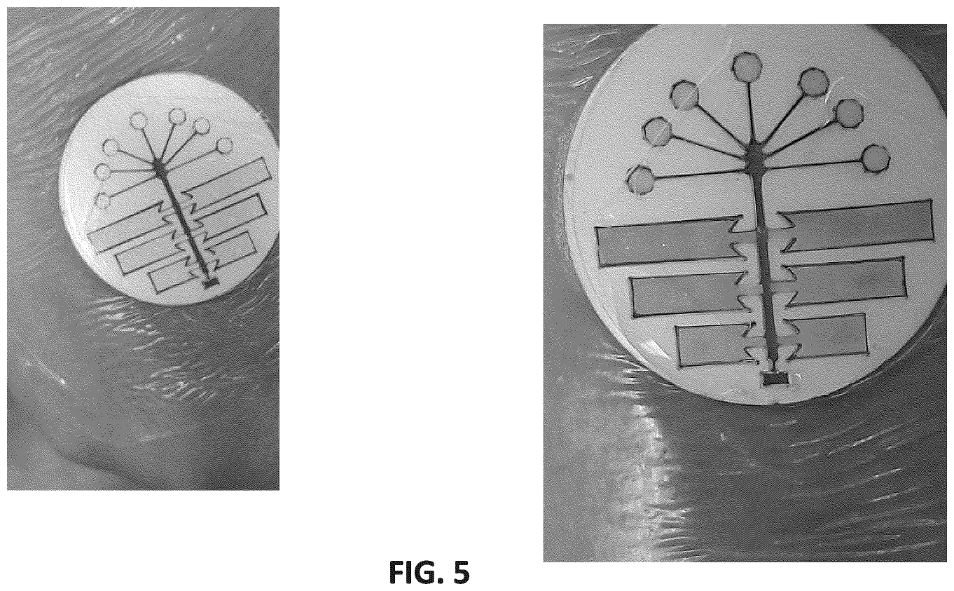

FIG. 5 illustrates a wearable device for sweat collection according to an embodiment of the present disclosure and illustrating a main channel, a vent, inlet channels, inlet ports and collection chambers. In contrast to the rectangular chambers in FIGS. 2-4, which were for removing air bubbles and terminated in a vent, the rectangular chambers in FIG. 5 are provided as rectangular collection chambers for collecting sweat. In an example embodiment, the collection chambers are designed so that they fill in a sequential manner, starting with the larger collection chambers, then moving to the next largest. In an implementation, depending on the flow rate, this can also be a time sequence, if gates are provided to determine, detect or record when the first sweat was coming in and then gate to measure another gate. This way, the device may be used to measure the flow rate of the sweat itself through the device or associated system. In an implementation, an approach may employ time points in the chip during the filling process. A vent is provided at the end of the set of collection chambers, to allow sweat to vent or evaporate out, to enable continuous flow through the device.

In use, sweat is collected in the sweat collection chambers, the device may be frozen to preserve the properties of the collected sweat. In an example implementation, the sweat collection chambers, or fibers provided therein, may be peeled out and sent off for mass spectroscopy analysis to analyze the sweat and detect biomarkers, such as metabolites of stress. When a subject undergoes stress tests, or sweat is collected during activity for example of an athlete, the samples may be collected and analyzed to determine what kind of biomarkers there are in different populations of people under different conditions.

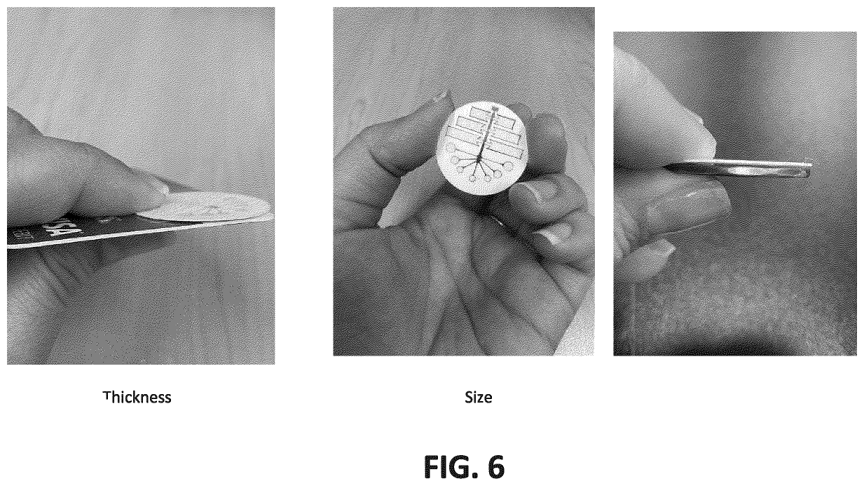

FIG. 6 illustrates side and top views of a wearable device according to an embodiment of the present disclosure to illustrate example thickness and size. In an example embodiment, the device has an annular or circular shape and is approximately 27 mm in diameter and 500 μm thick, or about the size of a Canadian two dollar coin. In an implementation, the device is attached to the user using a medical adhesive, for example on the underarm or on the side an arm, or another location from which sweat is to be collected. In an example implementation, the device may be replaceable and disposable, such that a new device is used each day for sweat collection. In an embodiment, the disposable device is in communication with electronics, for example such that the device may be adhered to and then peeled off the electronics.

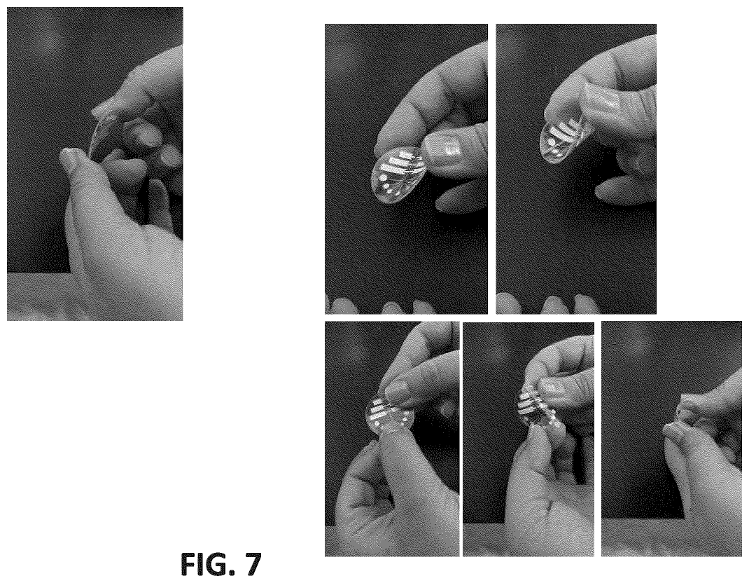

FIG. 7 illustrates a wearable device according to an embodiment of the present disclosure constructed of a flexible material. In an embodiment, the device comprises a transceiver, such as using a low power Bluetooth connection, to send information to a smart device or other processor. In another embodiment, the device itself may comprise one or more integrated circuits. In another embodiment, the wearable device according to an embodiment of the present disclosure is integrated into a garment, such as an athletic shirt or other garment with close adherence to the skin, for example a compression garment.

Embodiments of the present disclosure provide a wearable device that provides advantages to assist high performance athletes in training and development by measuring one or more biomarkers or stress. In another embodiment, the wearable device assists police personnel, or other front line or emergency response workers, to ensure that subjects are staying physically and mentally fit to perform their role. Subjects in such roles often deal with their managing stress loads appropriately, for example seeking to avoid high stress situations that may cause post-traumatic stress disorder (PTSD). In another implementation, a wearable device according to an embodiment of the present disclosure can assist with identifying symptoms or helping to manage PTSD or other stress-related conditions or illnesses. This can provide assistance to subjects who may, for example with PTSD, have an abnormal stress response, which can spiral out of control for them where they feel helpless, if mitigating actions are not taken.

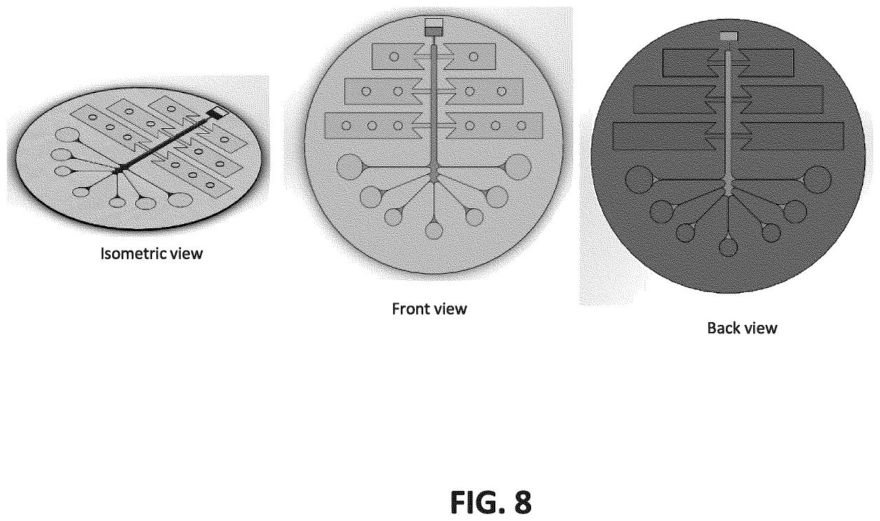

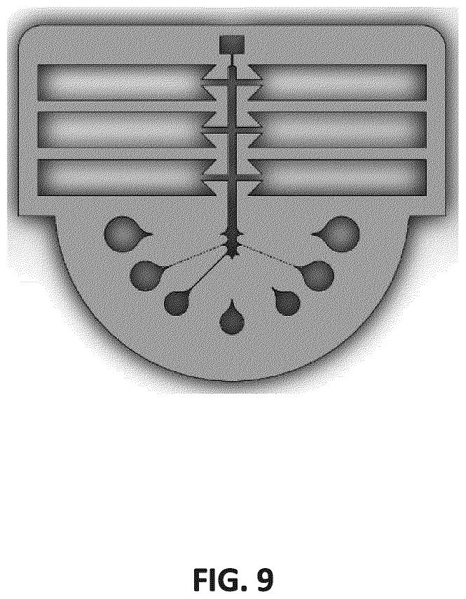

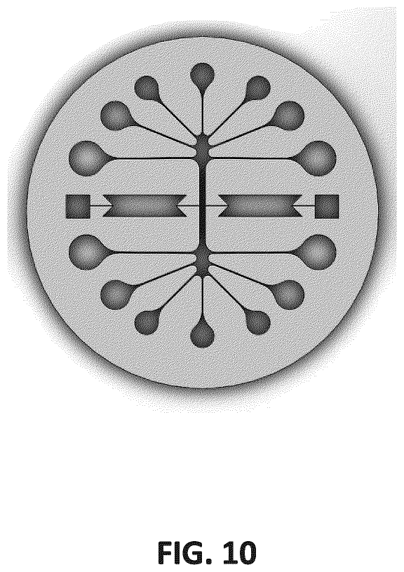

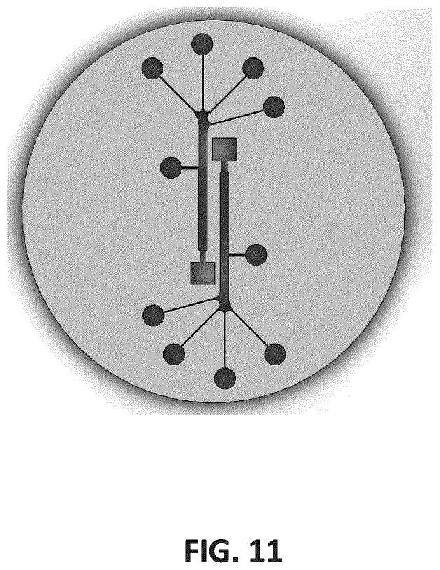

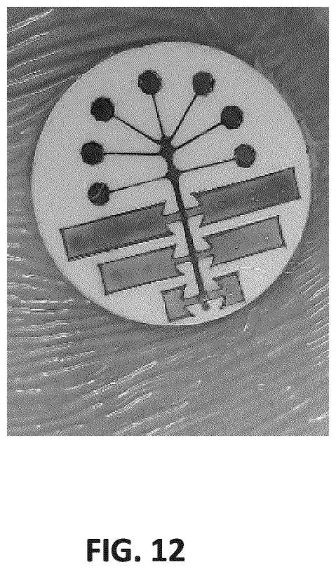

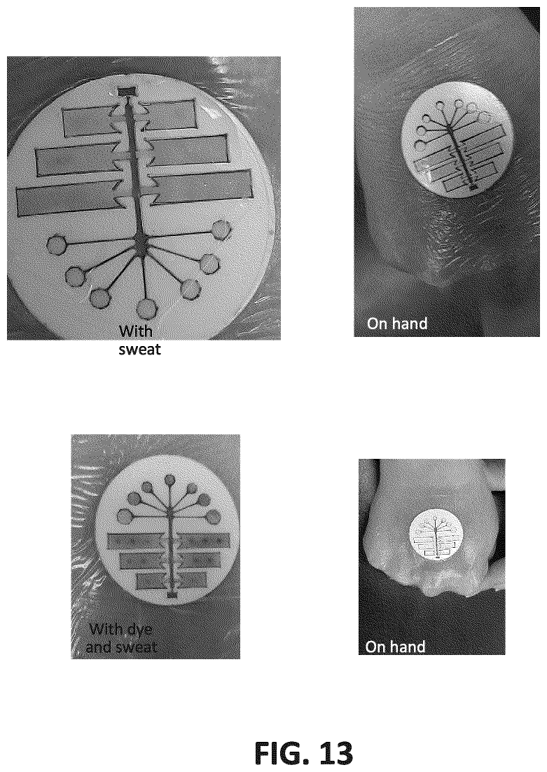

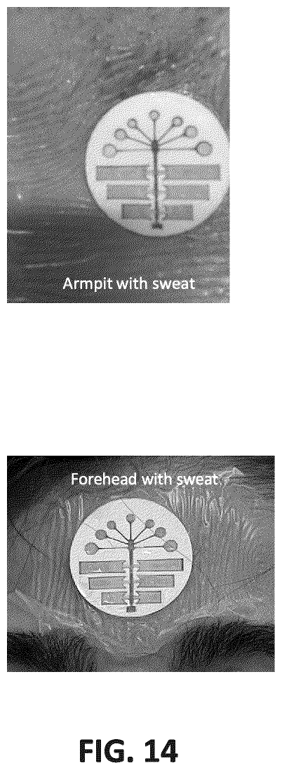

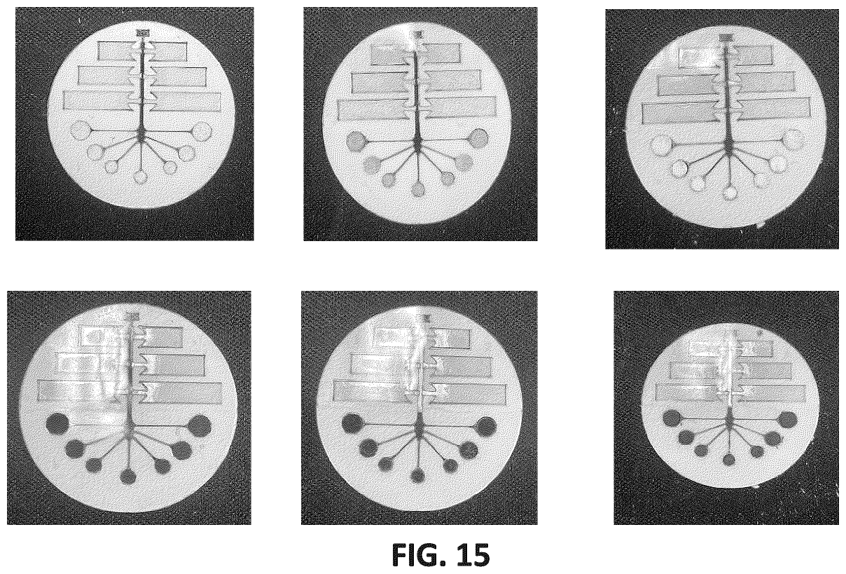

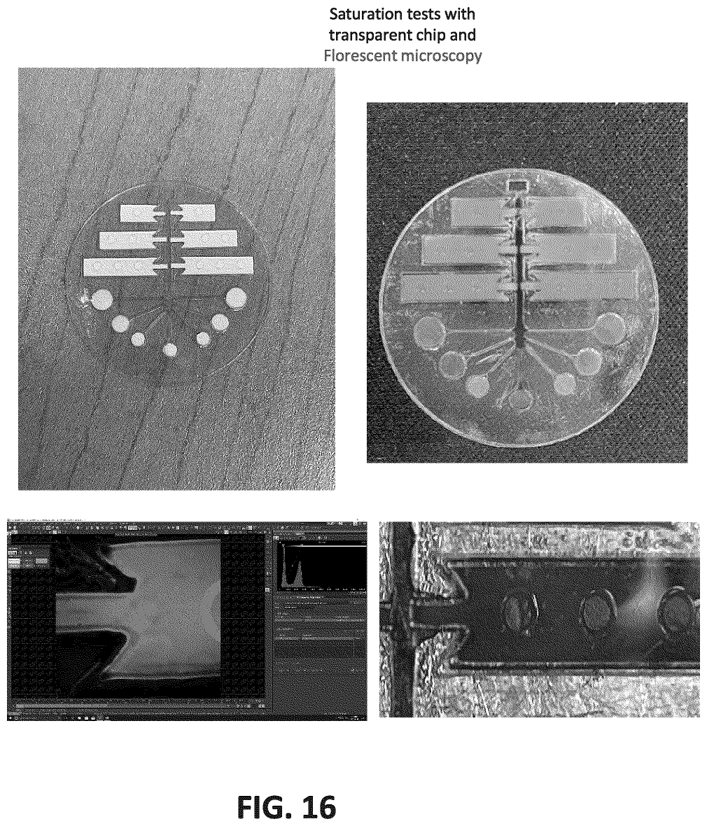

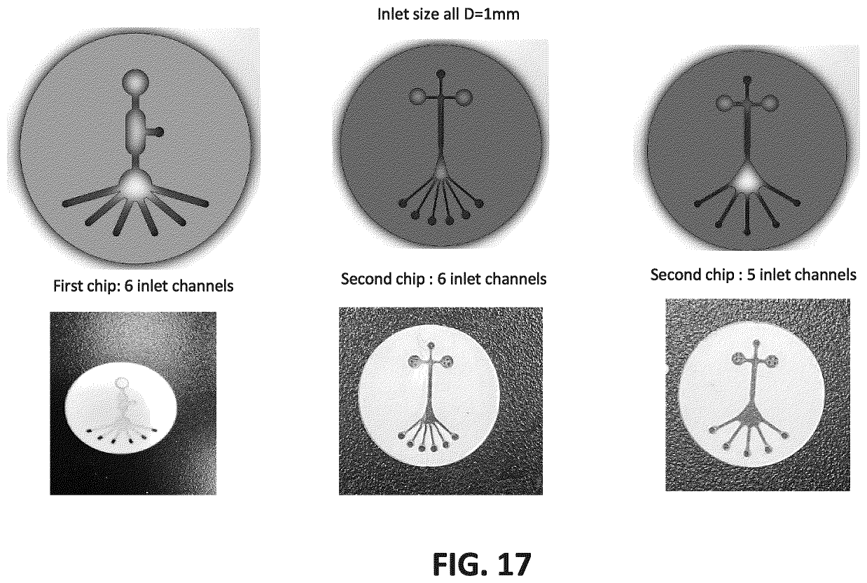

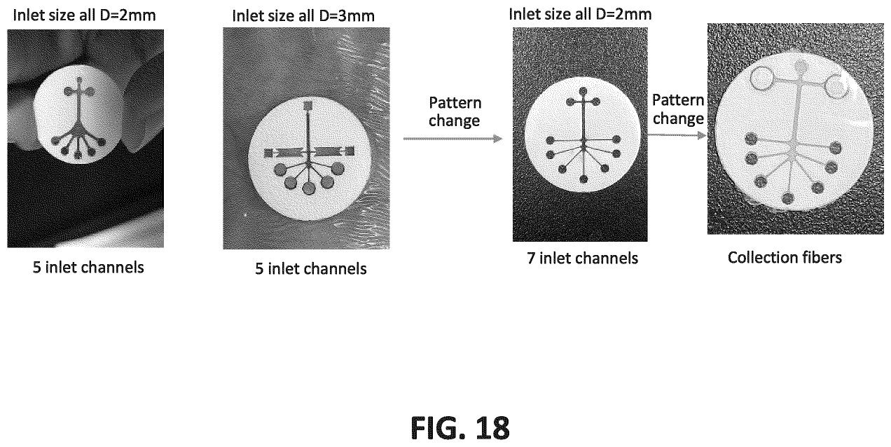

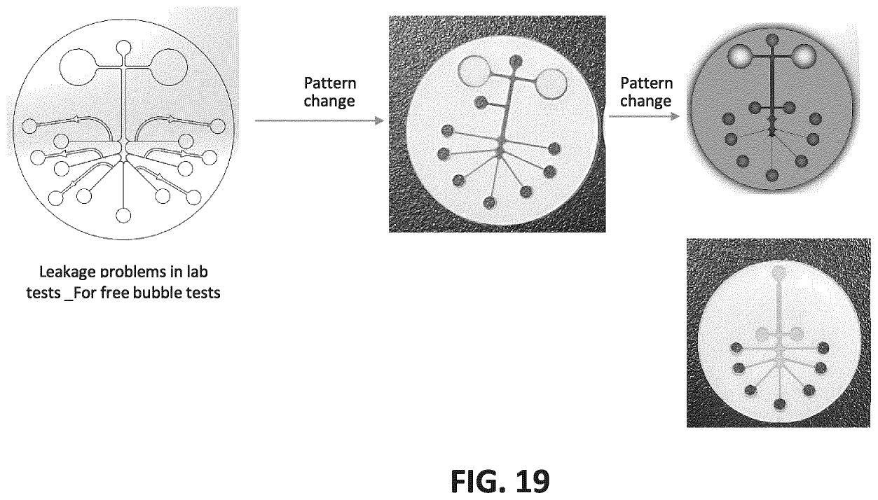

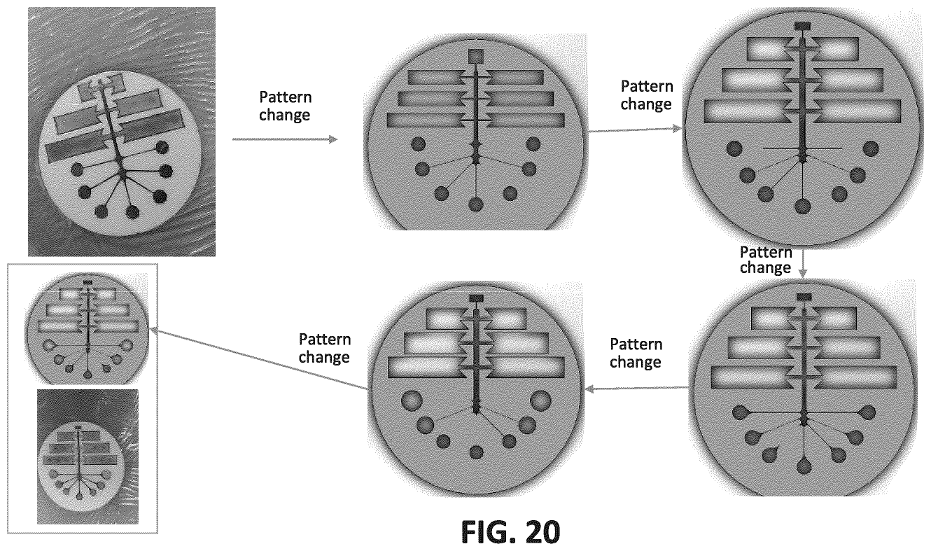

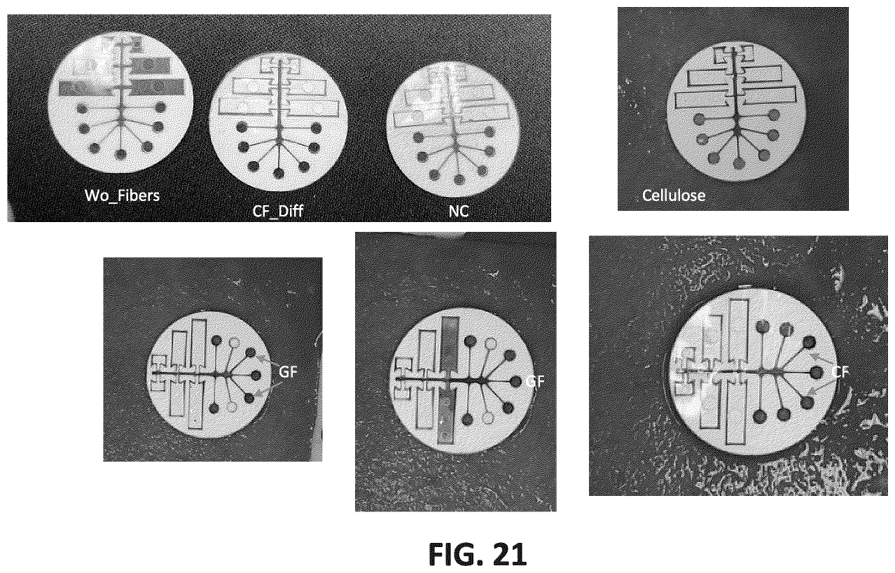

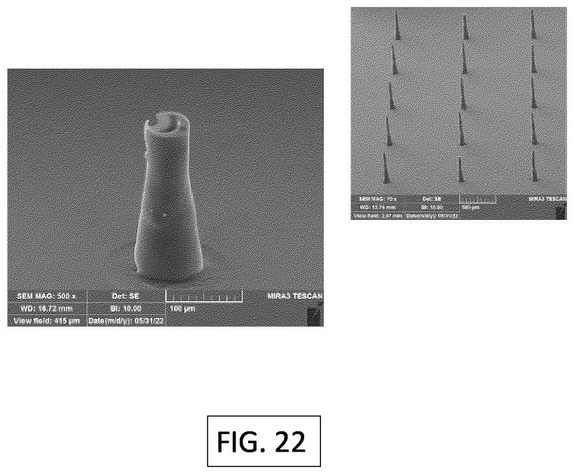

FIGS. 8-22 illustrate different example embodiments of wearable devices of the present disclosure, including different layouts, configurations, thicknesses, numbers of channels, and embodiments in use in various locations such as on a hand, in an armpit and on a forehead. The embodiments described and illustrated herein provide variation in different design elements, for example the shapes and the edges, to provide embodiments that may be geared towards affecting the filling of the collection channels, or getting rid of bubbles during sweat collection. Some embodiments of the device may provide a design with multiple inlets on top and bottom of the chip to enable collecting samples from multiple skin surfaces. Some embodiments of the device may provide a smooth fluidic flow through the device, and include an outlet vent to provide a continuous flow or multiple parallel lines of reaction chambers patterned onto the chip side by side and exposed to flow of analytes on different biomolecules receptors, allowing simultaneous detection of multiple analytes within a small design. In an example embodiment in which the wearable device comprises a sensor, the sensor is configured to sense a continuous flow across the sensor, to observe a time series rather than just a static moment in time.

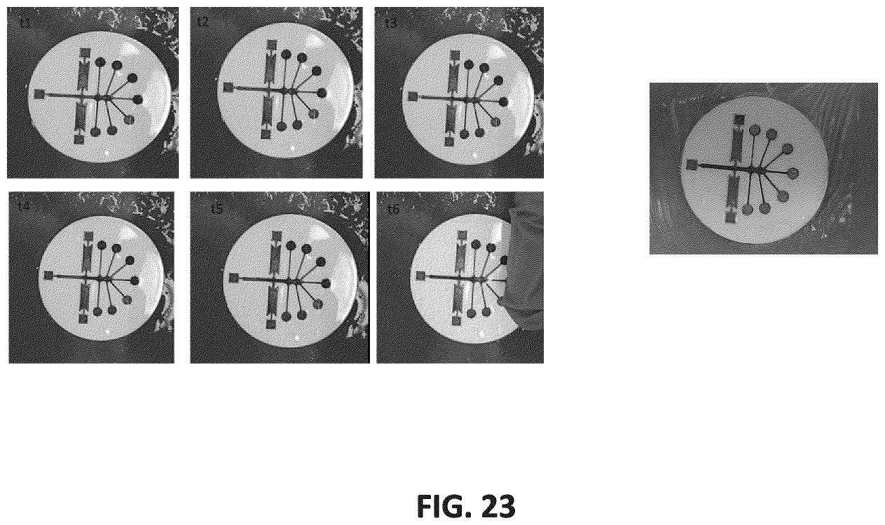

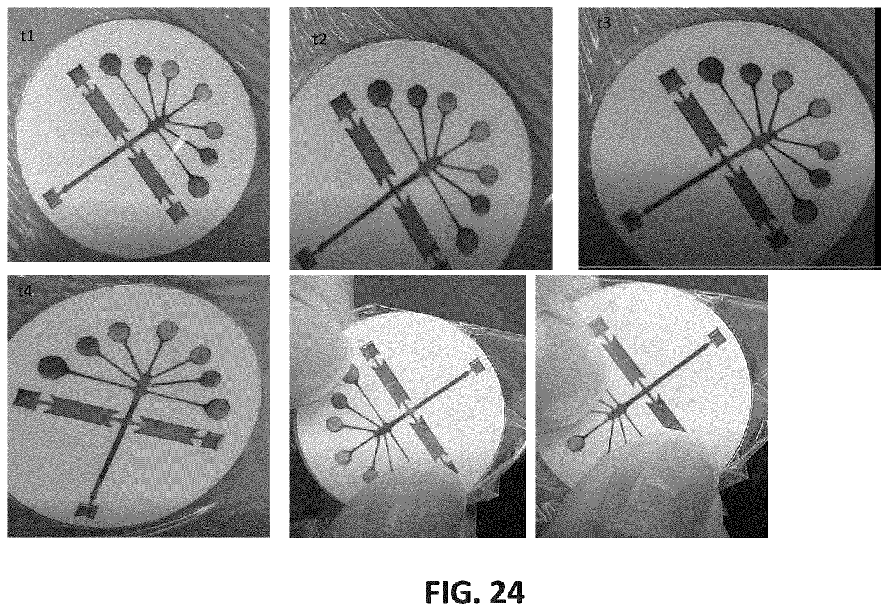

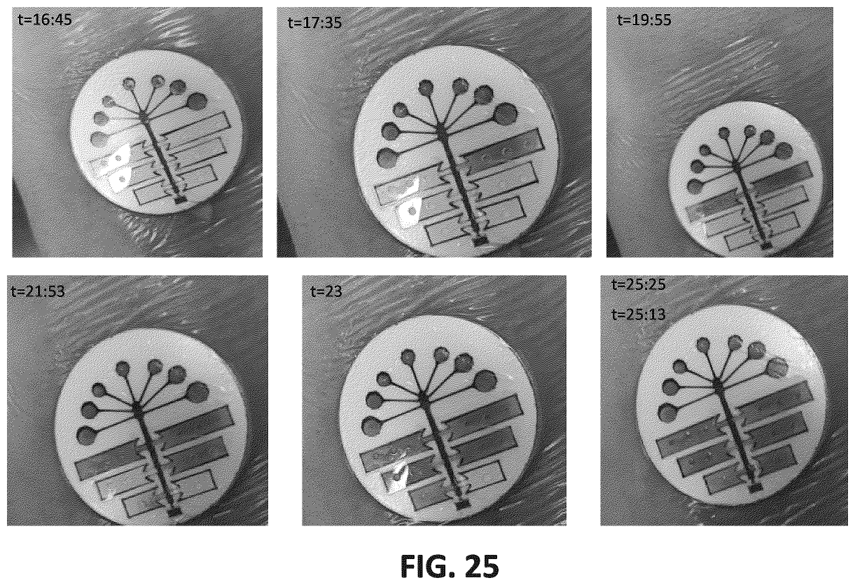

FIGS. 23-25 illustrate sequential filling of inlet ports and/or collection chambers in wearable devices according to embodiments of the present disclosure. In FIGS. 23-25, the rectangular collection channels are in communication with a box- or square-shaped vent for removing air bubbles. In the embodiment of FIG. 25, there are two sets of three rectangular collection chambers, on each side of the main channel, each collection chamber comprising fibers for collecting samples that can be removed and analyzed later. FIGS. 23-25 illustrate embodiments in which a dye has been introduced, so that the portion turns blue when the sweat is collected in the channels, or in filaments or other fibers provided within the collection chambers, with the collected sweat being visible in the different inlet ports. In an example embodiment, the inlet ports and collection chambers fill sequentially in a specific manner, where the first two collection chambers of a similar size get filled first, and then the next two of a next smaller size, and then the last pair are filled, as shown by both the dye progression and the associated time values.

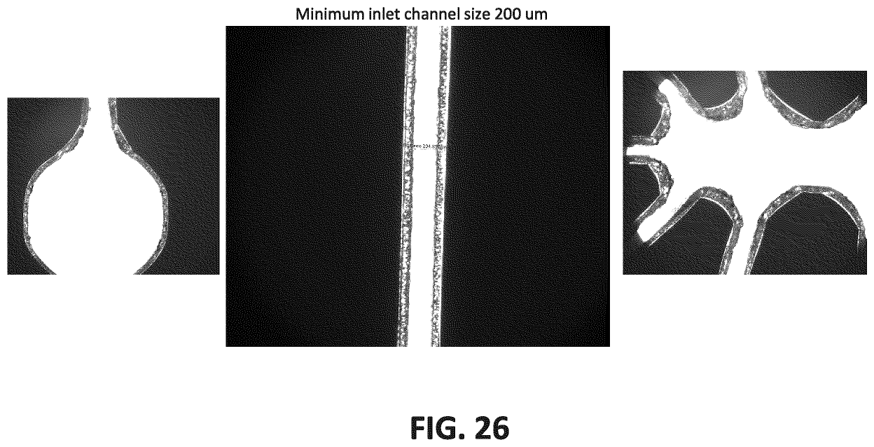

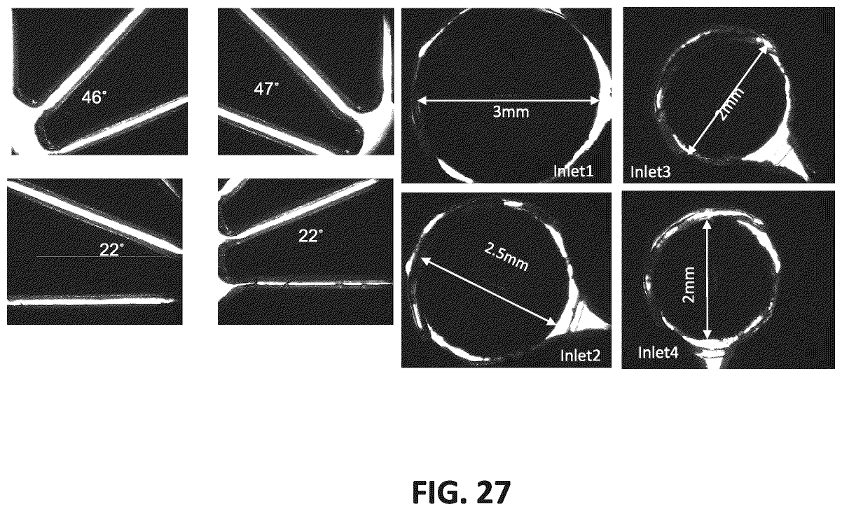

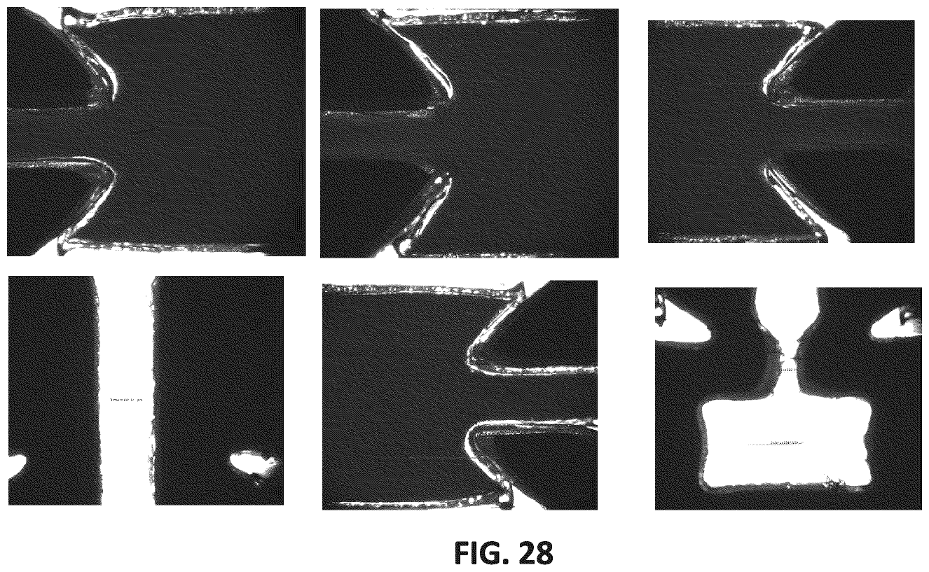

FIGS. 26-28 illustrate optical microscopy of different portions or segments of a wearable device according to embodiments of the present disclosure. These figures represent an example implementation in which a laser cutter was optimized for speed, power and run. Details and dimensions of example embodiments are indicated in FIGS. 26-28.

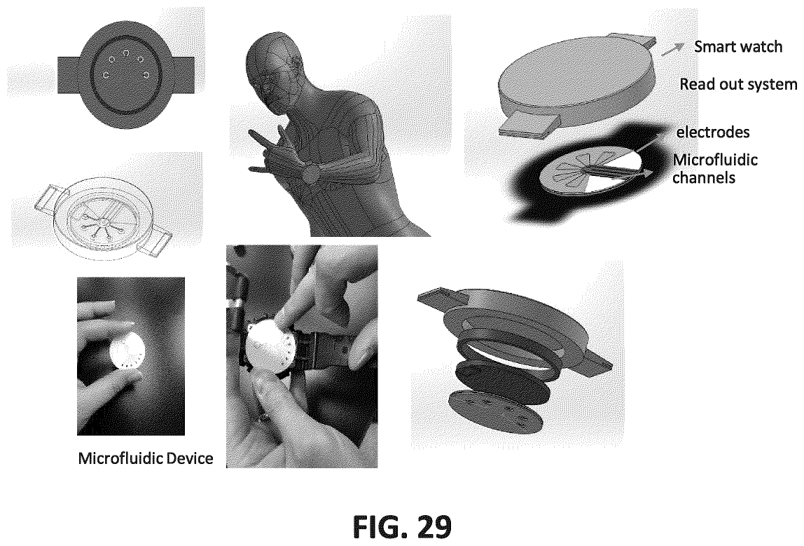

FIG. 29 illustrates an example implementation of a wearable device according to an embodiment of the present disclosure integrated into a smart watch. According to an example embodiment, the device is configured to assist with metabolite discovery to identify, in sweat, metabolites for stress and other medical conditions. According to another example embodiment, depending on the biomarkers or metabolites, the device may be integrated into another apparatus or device. For example, the wearable device according to an embodiment of the present disclosure may be integrated into a smart device, such as a smart watch, and may be configured to use biosensors, such as including one or more of optical, electrochemical, immunosensors etc., for example to use basic spectroscopy to detect one or more biomarkers or metabolites of interest, and optionally add color changes.

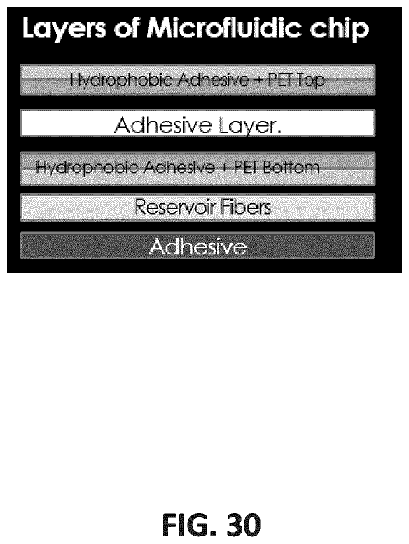

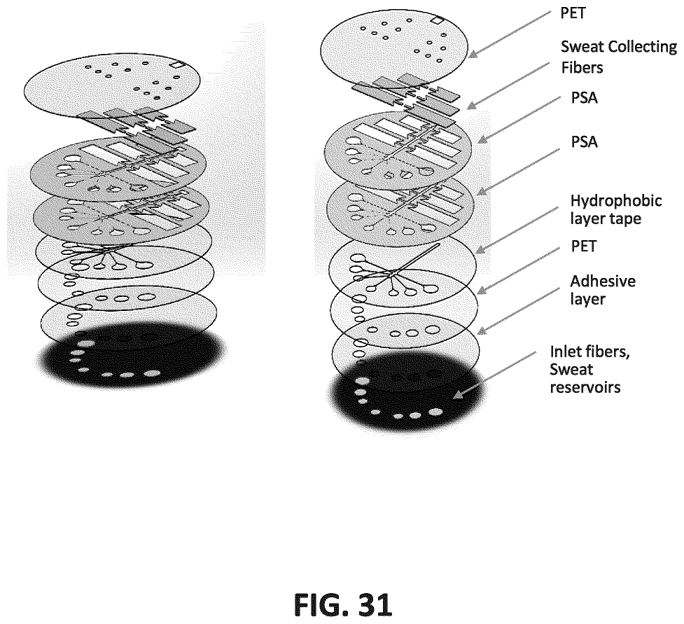

FIGS. 30 and 31 illustrate layers of manufacture of a wearable device according to example embodiments of the present disclosure. FIG. 30 illustrates a simplified block diagram representation of layers of manufacture of a wearable device according to an example embodiment, including inlet port fibers that are adhered to a subject's skin using an adhesive. An adhesive layer is sandwiched between hydrophilic top and bottom layers, which may be composed of a polyester or plastic resin such as polyethylene terephthalate (PET) and hydrophobic adhesive top and bottom layers where rectangular chambers and vents are located. FIG. 31 illustrates another embodiment of a more detailed exploded view of a wearable device, including how inlet fibers, sweat inlet ports, and sweat collecting fibers may be provided in relation to other layers such as PET or pressure sensitive adhesive (PSA).

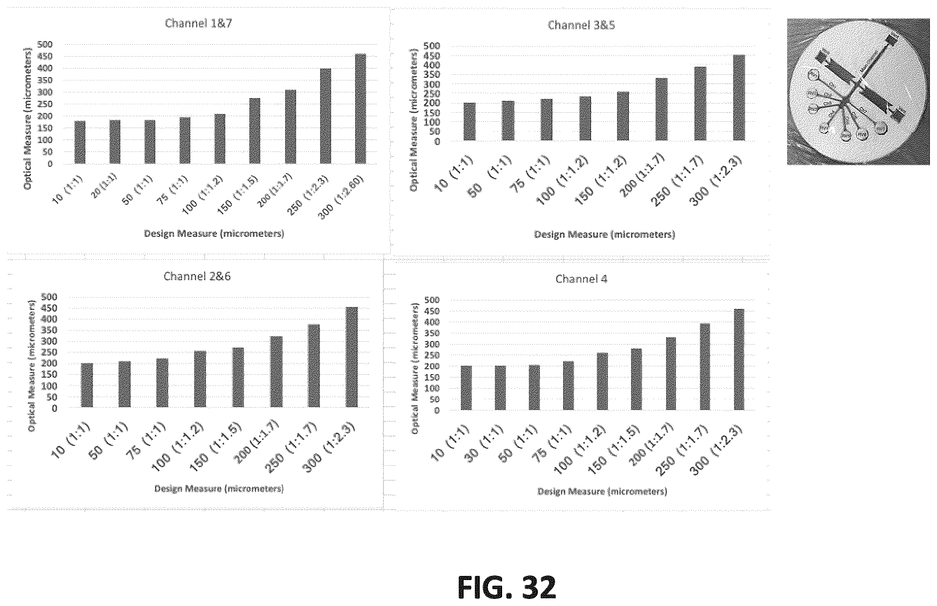

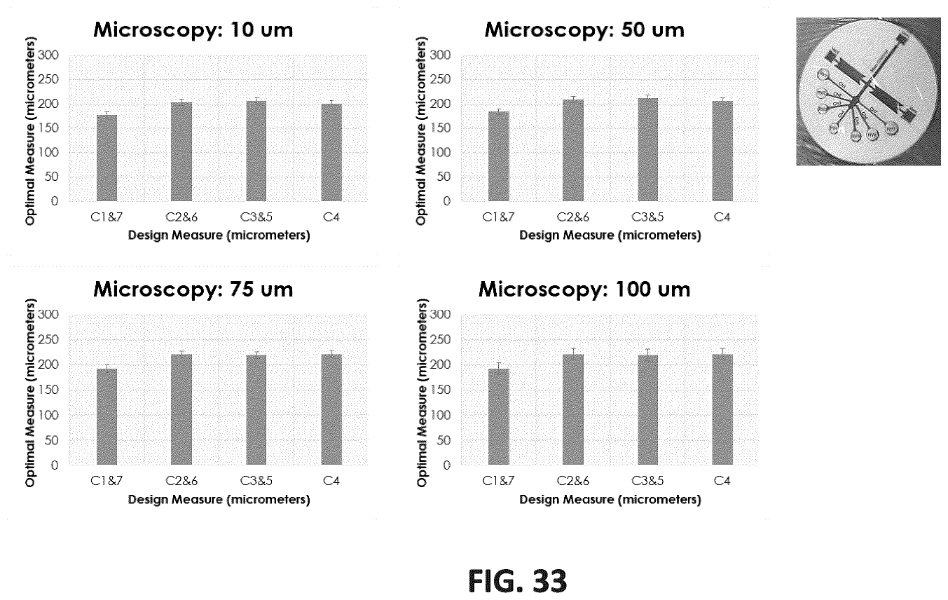

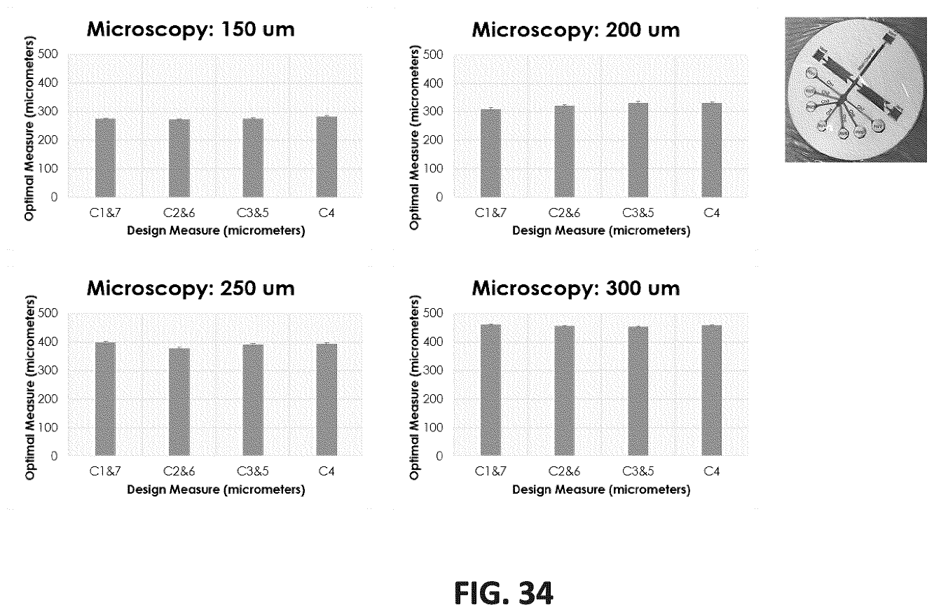

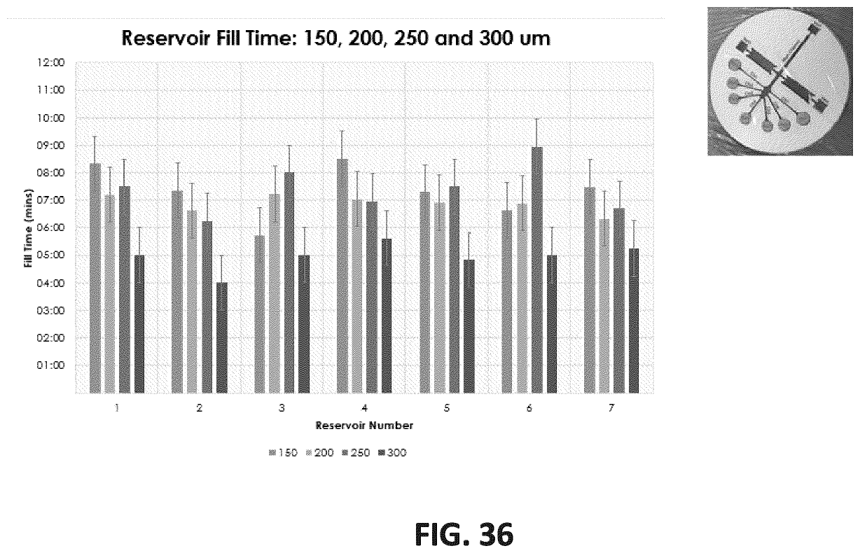

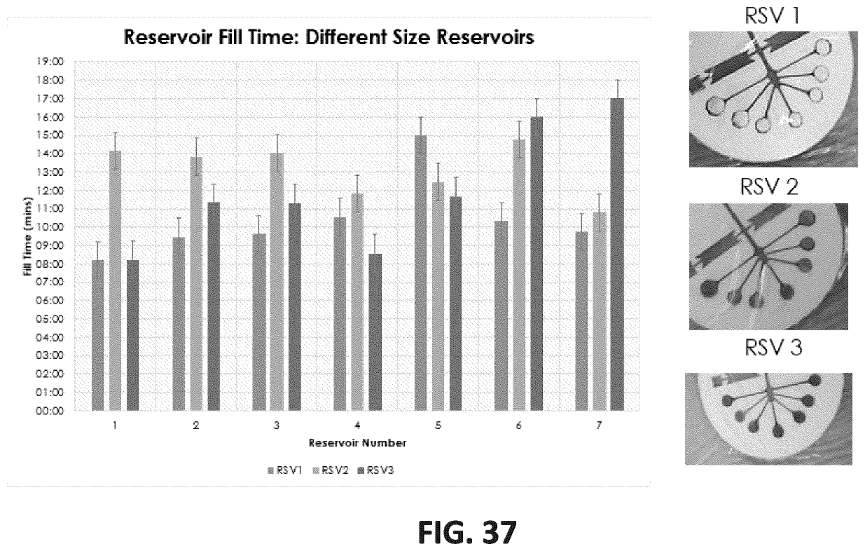

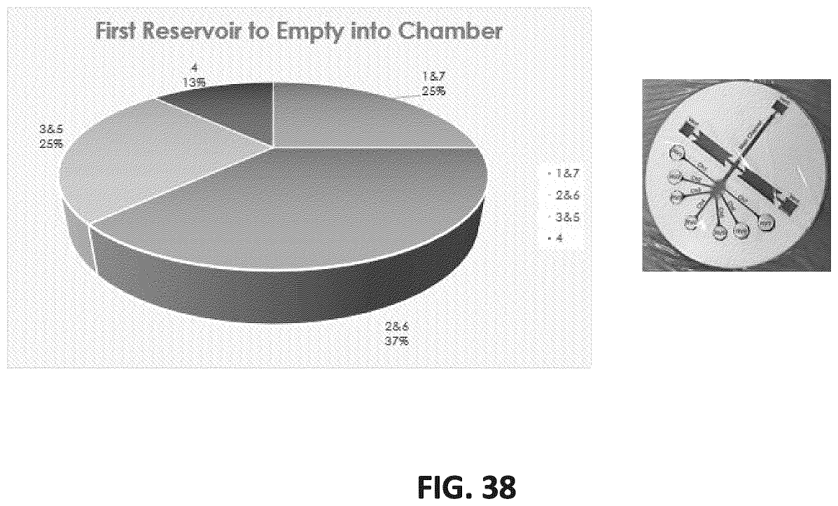

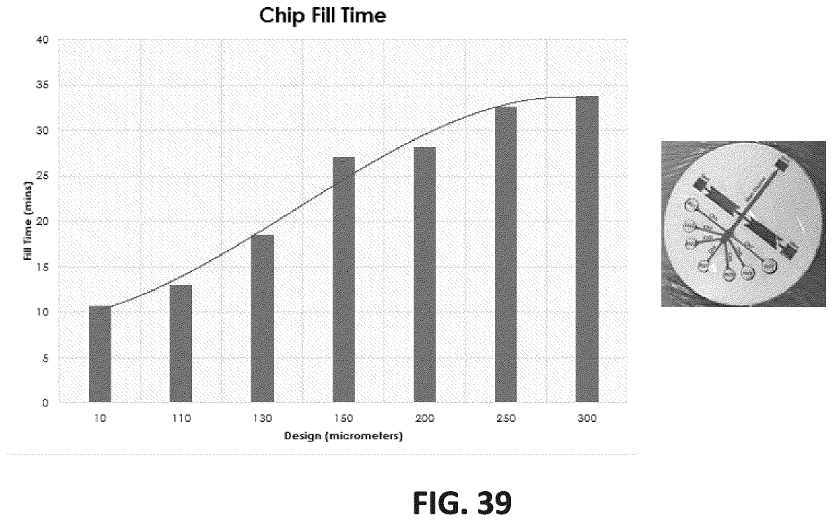

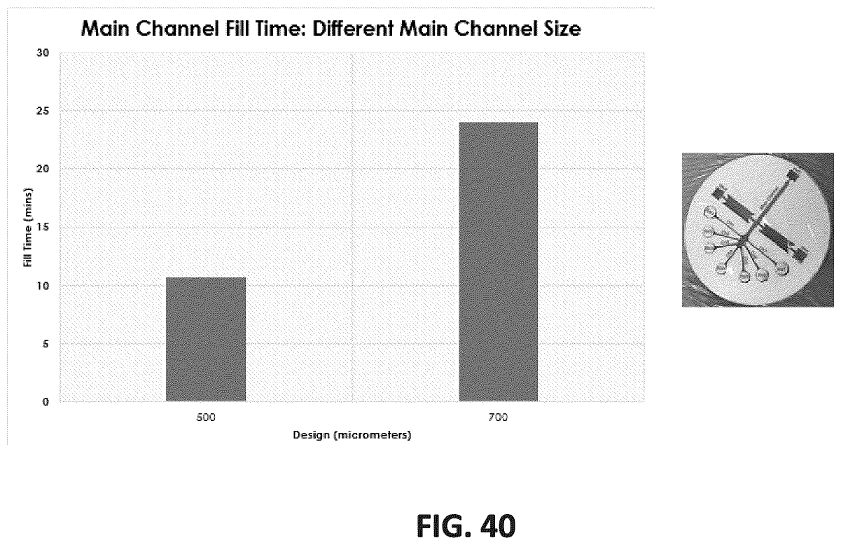

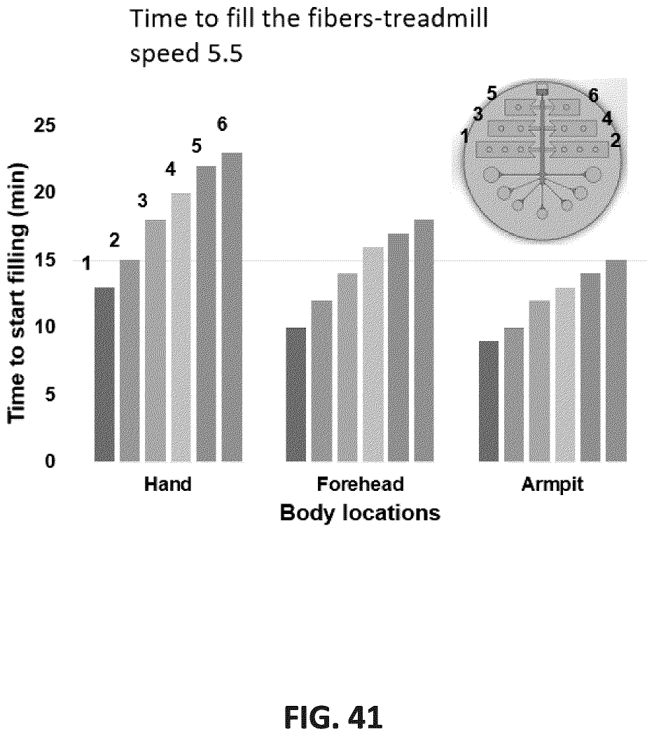

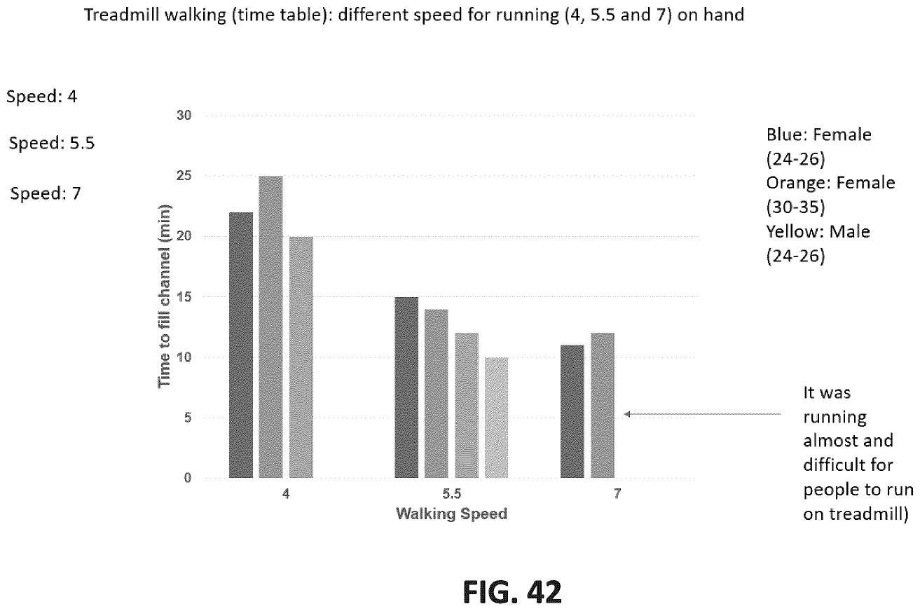

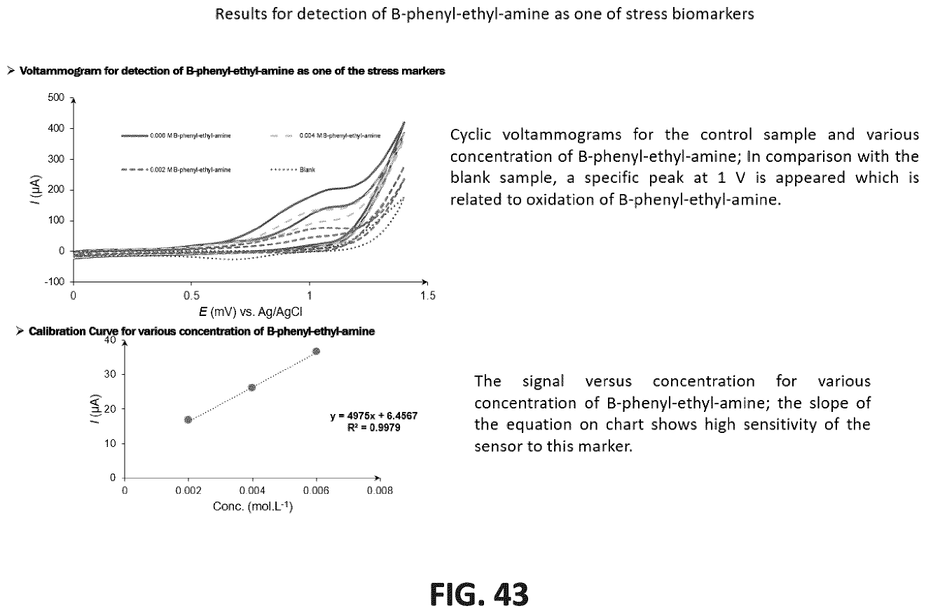

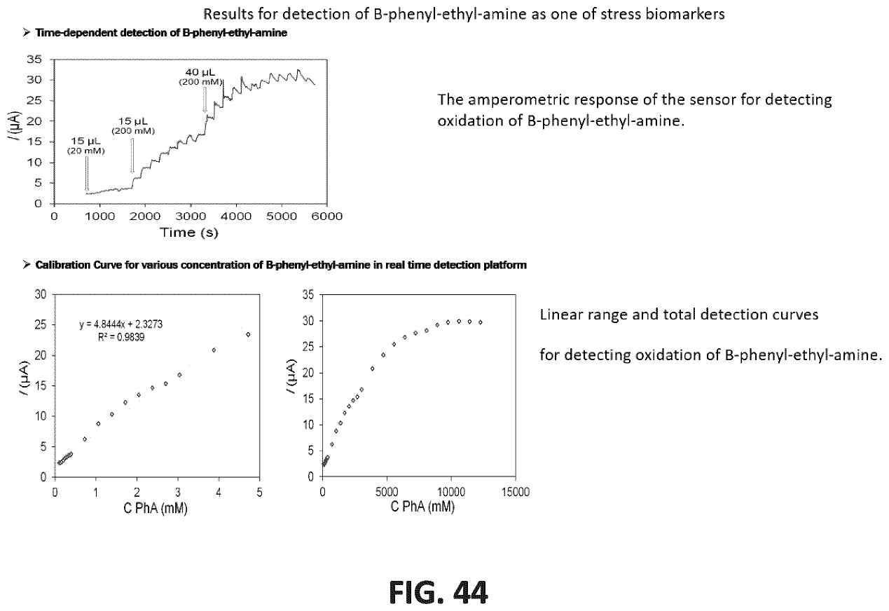

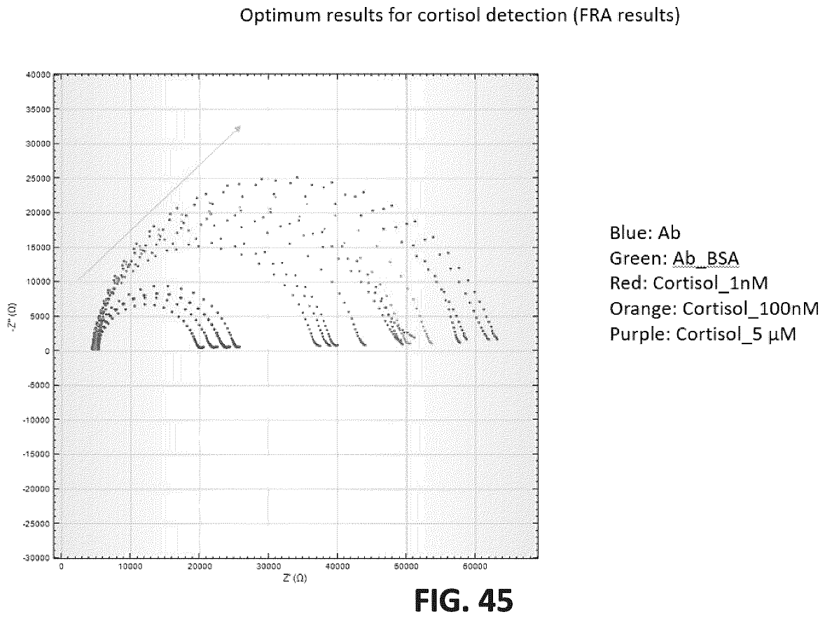

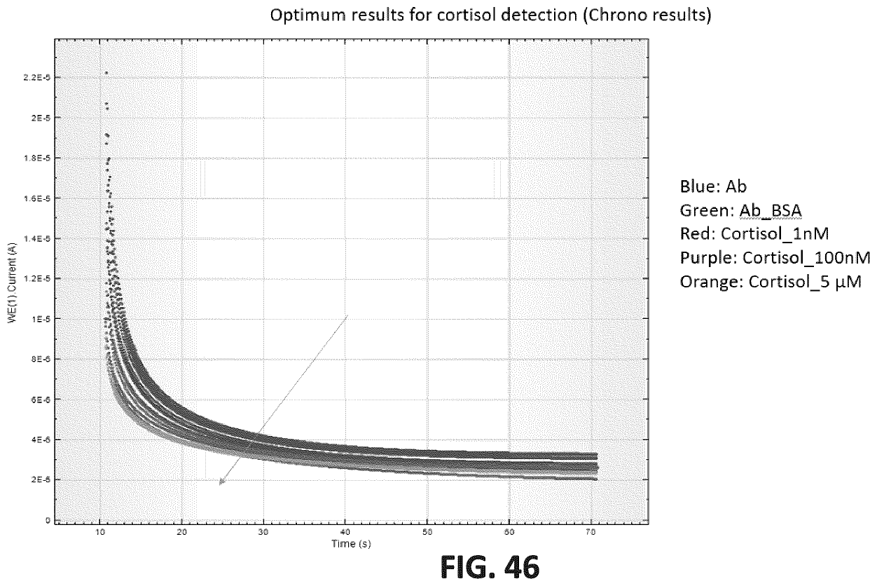

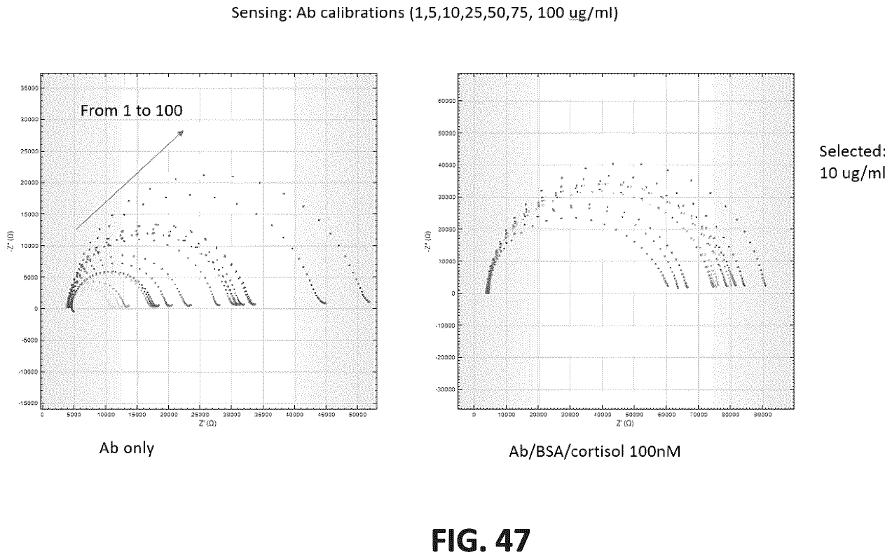

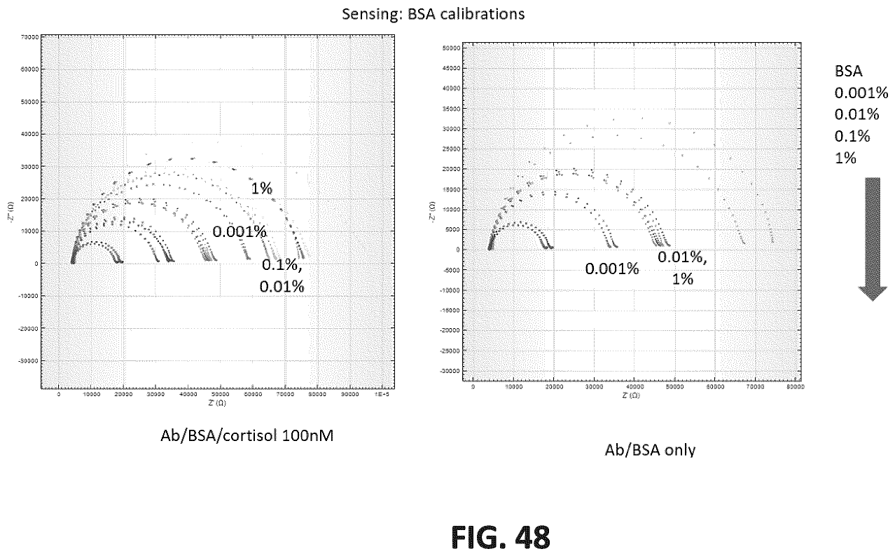

FIGS. 32-48 illustrate experimental results of implementation of wearable devices according to example embodiments of the present disclosure. The representations include indications of channel size, fill times for inlet ports and main channels (reaction chambers), first inlet port to empty, fill times for different body locations and at different running speeds, results for detection of biomarkers including B-phenyl-ethyl-amine, results for cortisol detection, and sensing for Ab and BSA calibrations. In an implementation, the Ab was for the sensor that was functionalized with a cortisol antibody, and BSA is bovine serum albumin which is used to look at non specific binding of the antibody. In another embodiment comprising a cortisol sensor, aptamers are used to functionalize it, since they are less expensive, have a smaller molecule size, and are more stable.

In relation to embodiments including a system configured for receiving, processing and providing data, performing methods, and providing functionality in relation to the measured and collected sweat and/or associated biomarker data, the system may include or be in communication with one or more computing platforms. Computing platform(s) may be configured to communicate with one or more remote platforms according to a client/server architecture, a peer-to-peer architecture, and/or other architectures. Remote platform(s) may be configured to communicate with other remote platforms via computing platform(s) and/or according to a client/server architecture, a peer-to-peer architecture, and/or other architectures. Users may access system via remote platform(s).

Computing platform(s) may be configured by machine-readable instructions. Machine-readable instructions may include one or more instruction modules. The instruction modules may include computer program modules configured to provide the functionality as described and illustrated herein.

In some embodiments, computing platform(s), remote platform(s), and/or external resources may be operatively linked via one or more electronic communication links. For example, such electronic communication links may be established, at least in part, via a network such as the Internet and/or other networks. It will be appreciated that this is not intended to be limiting, and that the scope of this disclosure includes implementations in which computing platform(s), remote platform(s), and/or external resources may be operatively linked via some other communication media.

A given remote platform may include one or more processors configured to execute computer program modules. The computer program modules may be configured to enable an expert or user associated with the given remote platform to interface with system and/or external resources, and/or provide other functionality attributed herein to remote platform(s). By way of non-limiting example, a given remote platform and/or a given computing platform may include one or more of a server, a desktop computer, a laptop computer, a handheld computer, a tablet computing platform, a NetBook, a Smartphone, a gaming console, and/or other computing platforms including smartphones, smart watches or other wearable devices.

External resources may include sources of information outside of system, external entities participating with system, and/or other resources. In some embodiments, some or all of the functionality attributed herein to external resources may be provided by resources included in system.

Computing platform(s) may include electronic storage, one or more processors, and/or other components. Computing platform(s) may include communication lines, or ports to enable the exchange of information with a network and/or other computing platforms. Computing platform(s) may include a plurality of hardware, software, and/or firmware components operating together to provide the functionality attributed herein to computing platform(s). For example, computing platform(s) may be implemented by a cloud of computing platforms operating together as computing platform(s).

Electronic storage may comprise non-transitory storage media that electronically stores information. The electronic storage media of electronic storage may include one or both of system storage that is provided integrally (i.e., substantially non-removable) with computing platform(s) and/or removable storage that is removably connectable to computing platform(s) via, for example, a port (e.g., a USB port, a firewire port, etc.) or a drive (e.g., a disk drive, etc.). Electronic storage may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage may include one or more virtual storage resources (e.g., cloud storage, a virtual private network, and/or other virtual storage resources). Electronic storage may store software algorithms, information determined by processor(s), information received from computing platform(s), information received from remote platform(s), and/or other information that enables computing platform(s) to function as described herein. In an implementation, block-chains may be used to protect personal data and allow the data to be used for research.

Embodiments of the present disclosure provide a microfluidic device comprising biocompatible materials and scalable technology enabling reliable collection of sweat during a physical activity. The device can collect human sweat of a practical volume within a practical amount of time when a person wearing the device is running on a treadmill, or performing some other type of physical activity. In embodiments comprising larger channel sizes, the chips became more uniform in size and filling. Embodiments of the present disclosure are configured to optimize the inlet ports and channels to provide a more uniform and reproducible sample collection. Embodiments of the present disclosure that are integrated with wearable biosensors enable online stress sensing.

Apparatus and methods according to the present disclosure, may be implemented and highly compatible with applications in stress diagnostics, court cognitive tests, sport medicines, disease pre-diagnostics. The device in use can be attached to different locations on the body, such as forehead, wrist, hand, palm, arm, back, or armpit to monitor the sweat generated from different locations simultaneously while mounted in a headband, wristbands or as part of a textile. A multipoint sweat collection according to embodiments of the present disclosure help the patients with injuries on one part of the body.

Embodiments of the present disclosure can be applied with respect to sweat collection from humans or from other animals, such as mice. Embodiments of the present disclosure can be applied with respect to sweat biomarkers for both acute and chronic stress by multiplexing sensing electrodes. Embodiments of the present disclosure can be applied with respect to detecting toxic environment by instantly and/or in real-time monitoring of any toxic chemicals exposed to the device. Embodiments of the present disclosure can be applied with respect to detection of exosome in sweat for skin immunity function diagnostics. Embodiments of the present disclosure can be applied with respect to using sweat biomarkers for detection, management or treatment of a medical condition, for example breast cancer, bowl inflammatory disease, infectious disease such as Tuberculosis, Parkinson, ALS, Schizophrenia, Diabetes mellitus, Cystic fibrosis, and chronic kidney failure.

Additional example embodiments are provided below:

Example embodiment 1: An apparatus for rapid and controllable sweat collection along with a label-free and accurate quantification of sweat composition, comprising:
a disposable, self-powered, and flexible wearable fluid device that is easily mountable and reliably sealable on the skin surface, enabling the collection of the sweat from multiple skin points and/or storing it within 1 nL-100 uL volume with preserved molecular contents with or without need for physical activities; a novel electrochemical biosensor with novel design and surface and with one or more electrodes, such as miniaturized, flexible, and multiplexed screen-printed electrodes, compatible with the flexible sweat-collecting fluid device, enabling continuous, longitudinal and/or single-point sensing of one or multiple target biomolecules and/or bioparticles in the collected sweat; and a miniaturized potentiostat readout device that is mounted inside a watch or a fitness band, and is easily assembled (a pick and place method) on the biosensor with the capability of Wireless and Bluetooth communications.

Example embodiment 2: The apparatus of example embodiment 1, wherein the fluid device is made of layers of flexible polymer and pressure sensitive adhesive sheets with different wettability properties, enabling reliable sweat collection generated in different rates in diverse populations.

Example embodiment 3: The apparatus of example embodiment 1, wherein the layers of the fluid device are compatible with scalable manufacturing technologies such as laser printing or injection molding, enabling rapid and highly reproducible production of the fluid device.

Example embodiment 4: The apparatus of example embodiment 1, wherein the layers of the fluid device can be easily aligned and bonded without a need for a press, plasma bonding, hot roller, or any expensive or bulky equipment for the assembly.

Example embodiment 5: The apparatus of example embodiment 1, wherein the polymer layers are made of medically approved grade polymers and can be used in close contact with the human skin.

Example embodiment 6: The apparatus of any one of example embodiments 1-4, wherein the fluid device contains several sweat collection ports (inlets) that can collect the sweat from multiple points on the skin while the fluid design of the device enables bubble-free sweat flow into the device, needed for further storage of the sweat or sensing of sweat composition.

Example embodiment 7: The apparatus of any one of example embodiments 1-6, wherein the combined wettability properties of all the layers in contact with the sweat enables autonomous (self-powered) flow of the sweat into the fluid device with desired flow rate and pattern without a need for any external pumping. This may be implemented regardless of sweat generation rate.

Example embodiment 8: The apparatus of any one of example embodiments 1-6, wherein the rapid collection of the sweat from the skin and flowing it into the fluid device eliminates the risk of sweat contamination with the environment or skin surface.

Example embodiment 9: The apparatus of example embodiment 1-6, wherein the outlet inlet ports of the fluid device inhibit the outflow of the sweat from the device and prevent collection of extra sweat into the device when it is saturated.

Example embodiment 10: The apparatus of any one of example embodiments 1 to 8, wherein the fluid device contains specific absorbing fibers with defined porosity, size, and thickness embedded at the downstream channels that enables storage of the collected sweat with preserved sweat composition, eliminating the usual drawback of concentrating the fluid composition after the collection.

Example embodiment 11: The apparatus of any one of example embodiments 1 to 8, wherein the fluid device contains optimized design of inlets, vents, openings, valves, and channel network that enables a sequential storage of the continuously collected sweat in different inlet ports, accessing the dynamics of sweat composition during its generation.

Example embodiment 12: The apparatus of any one of example embodiments 1 to 5, wherein the design of the fluid device with thin polymeric layers and versatile channel network enables sweat collection in diverse sweat generating populations with or without need for a physical activity.

Example embodiment 13: The apparatus of example embodiment 1, wherein the screen-printed electrodes assembled within the fluid device function as electrochemical biosensors that enable ultrasensitive sensitive and selective detection of the sweat composition within the fluid device.

Example embodiment 14: The apparatus of example embodiment 1, wherein optical biosensing can be used for non-contact and fast sensing and detections of sweat composition.

Example embodiment 15: The apparatus of any one of example embodiments 1 and 12, wherein the sensing electrode is coated on one of the flexible polymer substrates of the fluid device, therefore facilitating the assembly of the electrodes into the fluid device without a need for any specific insertion, alignment, or mounting signs.

Example embodiment 16: The apparatus of any one of example embodiments 1 to 15, wherein the design and size of the electrode system are compatible with all the components of the fluid device and therefore it does not have any adverse effect on the rate and pattern of sweat flow in the fluid device.

Example embodiment 17: The apparatus of any one of example embodiments 1 to 15, where three rigger electrodes placed across the sensing electrodes alarm the time the sweat reaches and covers the sensing electrodes and the time the fluid device is saturated with the sweat, enabling reliable sensing of the sweat at a right time, independent from the rate of sweat generation in diverse populations.

Example embodiment 18: The apparatus of any one of example embodiments 1 to 15, wherein the sweat composition can be detected in one or a combination of the continuous, longitudinal or single point measurement modes, depending upon the type of target biomolecules (metabolites, proteins, nucleic acids, dermal drugs) or bioparticles (microvesicles, exosomes, nanoparticles) detected in the sweat.

Example embodiment 19: The apparatus of any one of example embodiments 1 to 15, wherein the biosensors can detect enzymes and proteins in the sweat within the detection range of 1 pg/mL to 10 ng/ml, meeting the requirement for detection of many different proteins in the sweat.

Example embodiment 20: The apparatus of any one of example embodiments 1 to 18, wherein the fluid device with capillary fluid components of valves, soaked fibers, inlet ports, and fluid circuit design enables automation of several sensing steps, including incubation, washing, and sensing.

Example embodiment 21: The apparatus of any one of example embodiments 1 to 11, wherein the sensing electrodes are functionalized (read for binding of capture molecules) while they are screen printed, all in one single step, thereby eliminating the need for the multistep process of biosensor creation and improves the reproducibility and stability of the electrodes for long-term monitoring of sweat composition.

Example embodiment 22: The apparatus of example embodiment 20, wherein the bioreceptor (capture molecule) could be antibody, enzyme, or aptamer, enabling highly selective detection of target biomolecule or bioparticle in the sweat.

Example embodiment 23: The apparatus of any one of example embodiments 1 to 21, wherein the assembled fluid device and biosensor can simultaneously collect the sweat and detect multiple biomarkers.

Example embodiment 24: The apparatus of any one of example embodiments 1 to 21, wherein the small multichannel potentiostat readout embedded into a watch or a fitness band can be easily connected to the electrode using spring pins, record the electrical signals in real-time, analyze the signals, and send the results to a Cloud and web-App, enabling digital monitoring of the sweat.

Example embodiment 25: The apparatus of any one of example embodiments 1 to 24, wherein the assembled fluid device, biosensor, and digital readout can be worn on arms, hand, armpit, forehead, or in combination, enabling the comparison of the sweat composition among different skin surfaces.

Example embodiment 26: An apparatus of any one of example embodiments 1 to 25, where the multipoint sweat collection from the skin occurs simultaneous using multiple inlets located on different part of the skin without any risk of bubble generation in the fluid device as a result of delay in sweat generation in different parts of the skin.

Example embodiment 27: An apparatus of any one of example embodiments 1 to 16, wherein a layer of absorbing pad can be used to act as liquid inlet port and provide the device with large surface area of 706 cm² in contact with skin for sweat collection.

Example embodiment 28: An apparatus of any one of example embodiments 1 to 16, where the device is provided as a chip and is noninvasive and easily attached to the skin with no harm feeling even after wearing for several hours and easily detached from skin.

Example embodiment 29: An apparatus of any one of example embodiments 1 to 16, where the chip can collect sweat and can dynamically detect biomarkers in sweat in a sequential time points during sweat generation.

Example embodiment 30: An apparatus of any one of example embodiments 1 to 16, where the chip can differentiate the biomarkers concentrations in sweat generated from endocrine glands or apocrine glands.

Example embodiment 31: An apparatus of any one of example embodiments 1 to 16, where the sensing electrode is a layer of the device.

In the preceding description, for purposes of explanation, numerous details are set forth in order to provide a thorough understanding of the embodiments. However, it will be apparent to one skilled in the art that these specific details are not required. In other instances, well-known electrical structures and circuits are shown in block diagram form in order not to obscure the understanding. For example, specific details are not provided as to whether the embodiments described herein are implemented as a software routine, hardware circuit, firmware, or a combination thereof.

Embodiments of the disclosure can be represented as a computer program product stored in a machine-readable medium (also referred to as a computer-readable medium, a processor-readable medium, or a computer usable medium having a computer-readable program code embodied therein). The machine-readable medium can be any suitable tangible, non-transitory medium, including magnetic, optical, or electrical storage medium including a compact disk read only memory (CD-ROM), digital versatile disk (DVD), Blu-ray Disc Read Only Memory (BD-ROM), memory device (volatile or non-volatile), or similar storage mechanism. The machine-readable medium can contain various sets of instructions, code sequences, configuration information, or other data, which, when executed, cause a processor to perform steps in a method according to an embodiment of the disclosure. Those of ordinary skill in the art will appreciate that other instructions and operations necessary to implement the described implementations can also be stored on the machine-readable medium. The instructions stored on the machine-readable medium can be executed by a processor or other suitable processing device, and can interface with circuitry to perform the described tasks.

The above-described embodiments are intended to be examples only. Alterations, modifications and variations can be effected to the particular embodiments by those of skill in the art without departing from the scope, which is defined solely by the claims appended hereto.

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

1. A wearable apparatus for collecting sweat from a subject for stress monitoring, the apparatus comprising:
a fluid collection device configured for direct contact with the surface or sublayers of the skin of a subject when the wearable apparatus is in use, the fluid collection device comprising:
a main channel configured to collect sweat from the surface or sublayer of the skin of the subject;
a plurality of inlet channels in communication with the main channel;
a plurality of inlet ports, each of the plurality of inlet ports being uniquely associated with one of the plurality of inlet channels, for storing a sample of the collected sweat and preserving molecular contents of the stored sweat;
a delay valve configured to ensure that sufficient sweat is collected to fill the main channel before liquid moves further into the next channel;
at least one stop valve configured to stop the liquid for a period of time after main channel is filled completely with sweat, and optionally complete sensing the sweat analytes and open valves for the sweat to be removed from the device and provides continuous flow into the device; and
at least one vent configured to draw air bubbles out of the sweat collected in the main channel, to assist in seat collection and preserving the molecular contents of the stored sweat.

2. The wearable apparatus of claim 1 wherein the fluid collection device further comprises:
a plurality of collection chambers configured to collect and absorb the collected sweat for storage for later analysis.

3. The wearable apparatus of claim 2 wherein the plurality of collection chambers comprise removable fibers for collecting and absorbing the collected sweat for storage for removal for later analysis.

4. The wearable apparatus of claim 1 further comprising:
a biosensor in communication with the fluid collection device and configured to sense one or more biomarkers in the collected sweat.

5. The wearable apparatus of claim 4 wherein the biosensor is configured to sense one or more target biomolecules or bioparticles.

6. The wearable apparatus of claim 4 wherein the biosensor is a printed on the wearable apparatus and configured to sense the collected sweat from channel reaction chamber associated with the main channel.

7. The wearable apparatus of claim 1 further comprising:
a high aspect ratio microneedle array with hydrophilic conical and hollow needles and configured to continuously absorb the subsurface humidity of the skin.

8. A wearable sweat collection and analysis system comprising:
the wearable apparatus of claim 4; and
a potentiostat readout device in communication with the biosensor and configured to record electrical signals from the biosensor and to analyze the signals to enable digital monitoring of the sweat.

9. An apparatus for sweat collection and quantification of sweat composition, comprising:
a wearable fluid device configured for direct contact with a skin surface or for collecting subsurface humidity of the skin using a microneedle array, and configured to collect of sweat from multiple skin points or store the collected sweat in small volumes and with preserved molecular contents; and
a biosensor comprising a plurality of electrodes in communication with the wearable sweat-collecting fluid device, configured to sense one more target biomolecules and/or bioparticles in the collected sweat.

10. A system for sweat collection and quantification of sweat composition, comprising:
a wearable fluid device configured for direct contact with a skin surface, and configured to collect of sweat from multiple skin points or store the collected sweat in small volumes and with preserved molecular contents; and
a biosensor comprising a plurality of electrodes in communication with the wearable sweat-collecting fluid device, configured to sense one more target biomolecules and/or bioparticles in the collected sweat; and
a potentiostat readout device in communication with the biosensor and comprising a transceiver for communicating data associated with an output the biosensor electrodes.

11. A system for rapid and controllable sweat collection along with a label-free and accurate quantification of sweat composition, comprising:
a disposable, self-powered, and flexible wearable fluid device that is easily mountable and reliably sealable on a skin surface, enabling the collection of the sweat from multiple skin points and/or storing it within 1 nL-100 uL volume with preserved molecular contents with or without need for physical activities;
an electrochemical biosensor with miniaturized, flexible, and multiplexed screen-printed electrodes, compatible with the flexible sweat-collecting fluid device, enabling continuous, longitudinal and/or single-point sensing of one or multiple target biomolecules and/or bioparticles in the collected sweat; and
a miniaturized potentiostat readout device that is mounted inside a watch or a fitness band, and is easily assembled on the biosensor with communication capability.

12. A wearable sweat collection and analysis system comprising:
the wearable apparatus of claim 5; and
a potentiostat readout device in communication with the biosensor and configured to record electrical signals from the biosensor and to analyze the signals to enable digital monitoring of the sweat.

13. A wearable sweat collection and analysis system comprising:
the wearable apparatus of claim 6; and
a potentiostat readout device in communication with the biosensor and configured to record electrical signals from the biosensor and to analyze the signals to enable digital monitoring of the sweat.

14. A wearable sweat collection and analysis system comprising:
the wearable apparatus of claim 7; and
a potentiostat readout device in communication with the biosensor and configured to record electrical signals from the biosensor and to analyze the signals to enable digital monitoring of the sweat.

15. The wearable sweat collection and analysis system of claim 8 wherein the potentiostat readout device is configured to transmit results associated with analyzing the signals.

16. The wearable sweat collection and analysis system of claim 12 wherein the potentiostat readout device is configured to transmit results associated with analyzing the signals.

17. The wearable sweat collection and analysis system of claim 13 wherein the potentiostat readout device is configured to transmit results associated with analyzing the signals.

18. The wearable sweat collection and analysis system of claim 14 wherein the potentiostat readout device is configured to transmit results associated with analyzing the signals.
